(19) United States
(12) Patent Application Publication
(10) Pub. No.: US 2023/0211101 A1
(43) Pub. Date: Jul. 6, 2023

(54) LARYNGEAL MASK AIRWAY DEVICES

(71) Applicants: Oron ZACHAR, Tel Aviv (IL); AIRWAY MEDIX S.A., Warsaw (PL)

(72) Inventor: Oron ZACHAR, Tel Aviv (IL)

(21) Appl. No.: 18/000,709

(22) PCT Filed: Jun. 2, 2021
(86) PCT No.: PCT/IL2021/050653

§ 371 Date: Dec. 5, 2022

Related U.S. Application Data
(60) Provisional application No. 63/171,516, filed on Apr. 6, 2021.
(60) Provisional application No. 63/079,639, filed on Sep. 17, 2020.
(60) Provisional application No. 63/035,057, filed on Jun. 5, 2020.

(51) Int. Cl. A61M 16/04

(52) U.S. Cl. CPC A61M 16/045

(57) ABSTRACT

A laryngeal mask airway device (20, 320) is provided including an inflatable balloon (30, 330), which is (a) shaped so as to define at least an inflatable annular cuff (32, 332) and a proximal inflatable pharyngeal portion (34, 334), and (b) insertable through an anterior opening of a patients oral cavity. The inflatable balloon (30, 330) is configured such that when an inflatable chamber (56) thereof is filled with air at a pressure of 10 cm H2O and unconstrained, a volume of the air in a proximal one-third (60) of the inflatable chamber (56) equals at least 200% of a volume of the air in a distal one-third (62) of the inflatable chamber (56). Other embodiments are also described.

### CROSS-REFERENCE TO RELATED APPLICATIONS

The present application claims priority from U.S. Provisional Application 63/035,057, filed Jun. 5, 2020, U.S. Provisional Application 63/079,639, filed Sep. 17, 2020, and U.S. Provisional Application 63/171,516, filed Apr. 6, 2021, all of which are assigned to the assignee of the present application and incorporated herein by reference.

### FIELD OF THE APPLICATION

The present invention relates generally to lung ventilation devices, and specifically to laryngeal mask airway devices.

### BACKGROUND OF THE APPLICATION

Laryngeal mask airway devices are useful in facilitating lung ventilation by forming a low-pressure seal around the patient's laryngeal inlet, thereby avoiding the known harmful effects of endotracheal tube (ETT) devices, which form a seal within the trachea. Laryngeal mask airway devices have become standard medical devices, instead of ETT devices, for rapidly and reliably establishing an unobstructed airway in a patient in emergency situations and in the administration of anesthetic gases. Some laryngeal mask airway devices further include a drainage tube, which opens into the distal tip of the mask and emerges from the mouth of the patient.

During general anesthesia, pulmonary ventilation is secured with an ETT device or by a laryngeal mask airway device, and attention to the risk of complications related to a high intracuff pressure is important. When the cuff-to-tracheal wall pressure exceeds the tracheal capillary pressure (130-140 cm H2O) for approximately 15 minutes, the tracheal mucous membrane becomes ischemic. The intracuff pressure approximates the cuff-to-tracheal wall pressures in high volume/low pressure cuffs, and a cuff pressure below 120 cm H2O is recommended to prevent ischemic injury. In addition, recurrent laryngeal nerve palsy has been demonstrated in up to 5% of patients after intubation, and a high cuff pressure is suspected as contributing to this complication. Similarly, in patients provided with a laryngeal mask, a high cuff pressure may lead to palsy of the lingual, hypoglossal, and recurrent laryngeal nerves, and postoperative sore throat.

The risk during anesthesia with nitrous oxide (NO) is further complicated by the fact that NO gases penetrate the cuff, thereby gradually increasing the cuff pressure above the initial setting at which the cuff was inflated.

U.S. Pat. Nos. 8,783,256, 5,632,271, and 7,305,985, all to Brain, describe laryngeal mask airway devices.

Rokamp K Z et al., in “Tracheal tube and laryngeal mask cuff pressure during anaesthesia—mandatory monitoring is in need,” BMC Anesthesiology December 2010 10:20, describe “a prospective quality-control study,” in which “201 patients undergoing surgery during anaesthesia (without the use of nitrous oxide) were included for determination of the cuff pressure of the tracheal tubes and laryngeal masks” (abstract).

A “Quick Reference Guide,” Teleflex (Triangle Park, N.C., USA), 2013, lists various laryngeal mask airway Airways, having different sizes and maximum cuff volumes.

### SUMMARY OF THE APPLICATION

Some embodiments of the present invention provide a laryngeal mask airway device, which comprises an inflatable balloon, which is shaped so as to define at least an inflatable annular cuff and a proximal inflatable pharyngeal portion. The inflatable balloon is insertable through an anterior opening of an oral cavity of a patient. The inflatable annular cuff shaped so as to define a cuff anterior side that is configured to stabilize the inflatable annular cuff with respect to a laryngeal inlet of the patient when the inflatable balloon is disposed at an insertion location within a throat of the patient.

The laryngeal mask airway device further comprises a shaft, which is installed through the anterior opening of the oral cavity. The shaft has (a) a proximal end that is configured to be disposed outside the patient's oral cavity when the inflatable balloon is disposed at the insertion location, and (b) a distal end portion that is fixed to the inflatable balloon. The laryngeal mask airway device further comprises an airway tube, which passes through at least a longitudinal portion of the shaft, such as through an entirety of a length of the shaft. The proximal inflatable pharyngeal portion of the inflatable balloon forms an air-tight seal with an external surface of the shaft (such as by gluing or welding).

For some applications, the inflatable balloon is configured such that when an inflatable chamber thereof is filled with air at a pressure of 10 cm H2O and unconstrained, a volume of the air in a proximal one-third of the inflatable chamber equals at least 200% of a volume of the air in a distal one-third of the inflatable chamber, such as at least 300%, e.g., at least 350%, such as at least 400% of the volume of the air in the distal one-third of the inflatable chamber. Typically, the proximal inflatable pharyngeal portion of the inflatable balloon provides at least a portion of, such as all of, the proximal one-third of inflatable chamber.

By contrast, in convention laryngeal mask airway cuffs, the volume in the proximal one-third of the inflatable chamber of the cuff is approximately equal to the volume in the distal one-third of the inflatable chamber, when inflated at a pressure of 10 cm H2O, as well as at other pressures, including 20 and 25 cm H2O.

For some applications, the cuff anterior side, along a proximal side of the inflatable annular cuff, is shaped so as to define a posteriorly-recessed portion, i.e., a portion that is recessed in a posterior direction. Optionally, the posteriorly-recessed portion of the cuff anterior side is curved. The posteriorly-recessed portion prevents the inflatable annular cuff from applying excessive pressure to a posterior portion of the patient's tongue, which might cause tissue damage.

For some applications, an inflation tube of the laryngeal mask airway device comprises an inflation valve that is configured to be disposed outside the patient's oral cavity when the inflatable balloon is disposed at the insertion location, for holding a given inflation of the inflatable balloon. The laryngeal mask airway device is configured such that when the inflatable balloon, while the inflation valve is closed and the pressure within the inflatable balloon equals ambient pressure, is inserted from outside the oral cavity to the insertion location within the throat without opening the inflation valve, the pressure within the inflatable balloon increases from the ambient pressure to an inserted-location pressure, the inserted-location pressure less than 60 cm H2O, such as less than 45 cm H2O, e.g., less than 30 cm H2O.

There is therefore provided, in accordance with an Inventive Concept 1 of the present invention, a laryngeal mask airway device including:

an inflatable balloon, which is (a) shaped so as to define at least an inflatable annular cuff and a proximal inflatable pharyngeal portion, and (b) insertable through an anterior opening of an oral cavity of a patient, the inflatable annular cuff shaped so as to define a cuff anterior side that is configured to stabilize the inflatable annular cuff with respect to a laryngeal inlet of the patient when the inflatable balloon is disposed at an insertion location within a throat of the patient;

a shaft having (a) a proximal end that is configured to be disposed outside the patient's oral cavity when the inflatable balloon is disposed at the insertion location, and (b) a distal end portion that is fixed to the inflatable balloon;

an airway tube, which passes through at least a longitudinal portion of the shaft, and has (a) a proximal end that is configured to be disposed outside the patient's oral cavity when the inflatable balloon is disposed at the insertion location, and (b) a distal end that is open through the inflatable annular cuff, wherein the proximal inflatable pharyngeal portion of the inflatable balloon forms an air-tight seal with an external surface of the shaft; and

an inflation tube having (a) a proximal end that is configured to be disposed outside the patient's oral cavity when the inflatable balloon is disposed at the insertion location, and (b) a distal end that is coupled in fluid communication with an inflatable chamber defined by the inflatable balloon, for supplying air to the inflatable chamber,

wherein the inflatable balloon is configured such that when the inflatable chamber is filled with air at a pressure of 10 cm H2O and unconstrained, a volume of the air in a proximal one-third of the inflatable chamber equals at least 200% of a volume of the air in a distal one-third of the inflatable chamber,
the proximal one-third of the inflatable chamber extending distally from a proximal end of the inflatable chamber along one third of a length of the inflatable chamber,
the distal one-third of the inflatable chamber extending proximally from a distal end of the inflatable chamber along one third of the length of the inflatable chamber, and
the length measured along a straight line segment connecting (a) a distal centroid of the distal end of the inflatable chamber and (b) a proximal centroid of the proximal end of the inflatable chamber.
   Inventive Concept 2. The laryngeal mask airway device according to Inventive Concept 1, wherein the inflatable balloon is configured such that when the inflatable chamber is filled with the air at the pressure of 10 cm H2O and unconstrained, the volume of the air in the proximal one-third of the inflatable chamber equals at least 300% of the volume of the air in the distal one-third of the inflatable chamber.
   Inventive Concept 3. The laryngeal mask airway device according to Inventive Concept 2, wherein the inflatable balloon is configured such that when the inflatable chamber is filled with the air at the pressure of 10 cm H2O and unconstrained, the volume of the air in the proximal one-third of the inflatable chamber equals at least 350% of the volume of the air in the distal one-third of the inflatable chamber.
   Inventive Concept 4. The laryngeal mask airway device according to Inventive Concept 3, wherein the inflatable balloon is configured such that when the inflatable chamber is filled with the air at the pressure of 10 cm H2O and unconstrained, the volume of the air in the proximal one-third of the inflatable chamber equals at least 400% of the volume of the air in the distal two-third of the inflatable chamber.
   Inventive Concept 5. The laryngeal mask airway device according to Inventive Concept 1, wherein the inflatable balloon is configured such that when the inflatable chamber is filled with air at a pressure of 20 cm H2O and unconstrained, the volume of the air in the proximal one-third of the inflatable chamber equals at least 200% of the volume of the air in the distal one-third of the inflatable chamber.
   Inventive Concept 6. The laryngeal mask airway device according to Inventive Concept 1, wherein the inflatable balloon is configured such that when the inflatable chamber is filled with air at a pressure of 25 cm H2O and unconstrained, the volume of the air in the proximal one-third of the inflatable chamber equals at least 200% of the volume of the air in the distal one-third of the inflatable chamber.
   Inventive Concept 7. The laryngeal mask airway device according to Inventive Concept 1, wherein the inflatable balloon is configured such that when the inflatable chamber is filled with the air at the pressure of 10 cm H2O and unconstrained, the volume of the air in the proximal one-third of the inflatable chamber is at least 5 cc.
   Inventive Concept 8. The laryngeal mask airway device according to Inventive Concept 7, wherein the inflatable balloon is configured such that when the inflatable chamber is filled with the air at the pressure of 10 cm H2O and unconstrained, the volume of the air in the proximal one-third of the inflatable chamber is at least 10 cc.
   Inventive Concept 9. The laryngeal mask airway device according to Inventive Concept 1, wherein the inflatable balloon and the shaft are configured such that the proximal inflatable pharyngeal portion of the inflatable balloon forms the air-tight seal entirely around a longitudinal portion of shaft.
   Inventive Concept 10. The laryngeal mask airway device according to Inventive Concept 1, the proximal inflatable pharyngeal portion extends in both lateral directions away from the straight line segment.
   Inventive Concept 11. The laryngeal mask airway device according to Inventive Concept 10, wherein the proximal inflatable pharyngeal portion is reflectionally symmetric about a plane of symmetry that extends in an anterior/posterior direction, wherein the straight line segment falls in the plane of symmetry.
   Inventive Concept 12. The laryngeal mask airway device according to Inventive Concept 1, wherein the cuff anterior side is configured to form a seal around the laryngeal inlet of the patient when the inflatable balloon is disposed at the insertion location.
   Inventive Concept 13. The laryngeal mask airway device according to any one of Inventive Concepts 1-12,

wherein the inflation tube includes an inflation valve that is configured to be disposed outside the patient's oral cavity when the inflatable balloon is disposed at the insertion location, and

wherein the laryngeal mask airway device is configured such that when the inflatable balloon, while the inflation valve is closed and the pressure within the inflatable balloon equals ambient pressure, is inserted from outside the oral cavity of the patient to the insertion location within the throat without opening the inflation valve, the pressure within the inflatable balloon increases from the ambient pressure to an inserted-location pressure, the inserted-location pressure less than 60 cm H2O.

Inventive Concept 14. The laryngeal mask airway device according to Inventive Concept 13, wherein the inserted-location pressure is less than 45 cm H2O.
Inventive Concept 15. The laryngeal mask airway device according to Inventive Concept 14, wherein the inserted-location pressure is less than 30 cm H2O.
Inventive Concept 16. The laryngeal mask airway device according to any one of Inventive Concepts 1-12,

wherein the inflatable annular cuff is shaped so as to define a distal tip portion that includes a distal end of the inflatable annular cuff,

wherein the distal tip portion is configured to form a seal against an upper esophageal sphincter when the inflatable balloon is disposed at the insertion location, and

wherein a distal wall of the distal tip portion has a thickness of at least 2 mm.

Inventive Concept 17. The laryngeal mask airway device according to Inventive Concept 16, further including a drainage tube that (a) extends from a gastric channel having a distal gastric opening through the distal tip portion, and (b) has a proximal drainage port at location outside the patient's oral cavity when the inflatable balloon is disposed at the insertion location.
Inventive Concept 18. The laryngeal mask airway device according to any one of Inventive Concepts 1-12,

wherein the laryngeal mask airway device includes a distal tip portion located distal to the inflatable annular cuff, and

wherein the distal tip portion is configured to form a seal against an upper esophageal sphincter when the inflatable balloon is disposed at the insertion location.

Inventive Concept 19. The laryngeal mask airway device according to Inventive Concept 18,

wherein the laryngeal mask airway device includes an inner support, which is mostly covered by the inflatable balloon,

wherein the inflatable balloon is shaped so as to define a distal inflatable tip portion, which is in fluid communication with the rest of the inflatable chamber of the inflatable balloon, and

wherein the distal inflatable tip portion entirely surrounds a longitudinal portion of the distal tip portion.

Inventive Concept 20. The laryngeal mask airway device according to Inventive Concept 19, wherein a distal end of the longitudinal portion is at a distance from a distal-most tip of the distal tip portion, the distance between 1 and 5 mm.
Inventive Concept 21. The laryngeal mask airway device according to Inventive Concept 19, wherein the longitudinal portion has a length of between 1 and 5 cm.
Inventive Concept 22. The laryngeal mask airway device according to Inventive Concept 21, wherein the length is between 1 and 3 cm.
Inventive Concept 23. The laryngeal mask airway device according to Inventive Concept 21, wherein the length is between 2 and 4 cm.
Inventive Concept 24. The laryngeal mask airway device according to Inventive Concept 19, wherein the longitudinal portion has a greatest width, measured from side to side of the inflatable balloon, of no more than 20 mm along a length of the longitudinal portion.
Inventive Concept 25. The laryngeal mask airway device according to Inventive Concept 19, wherein the distal tip portion includes a distal-most non-balloon-covered tip portion, defined by the inner support.
Inventive Concept 26. The laryngeal mask airway device according to Inventive Concept 19, wherein a proximal end of the distal inflatable tip portion is at least partially coupled to the inner support, so as to restrict air flow between the distal inflatable tip portion and the inflatable annular cuff, without entirely blocking the air flow.
Inventive Concept 27. The laryngeal mask airway device according to Inventive Concept 19, further including a drainage tube that:

extends from a gastric channel that is defined by the inner support,

has a distal gastric opening through the distal tip portion, and

has a proximal drainage port at location outside the patient's oral cavity when the inflatable balloon is disposed at the insertion location.

Inventive Concept 28. The laryngeal mask airway device according to any one of Inventive Concepts 1-12, wherein the laryngeal mask airway device is configured such that when the inflatable chamber is filled with the air at the pressure of 10 cm H2O and unconstrained, a portion of the proximal inflatable pharyngeal portion of the inflatable balloon bulges posteriorly with respect to the external surface of the shaft.
Inventive Concept 29. The laryngeal mask airway device according to any one of Inventive Concepts 1-12, wherein a distal portion of the shaft passes through at least a portion of the proximal inflatable pharyngeal portion of the inflatable balloon, such that, when the inflatable chamber is filled with the air at the pressure of 10 cm H2O and unconstrained, a portion of the inflatable chamber defined by the proximal inflatable pharyngeal portion surrounds 360 degrees of the distal portion of the shaft.
Inventive Concept 30. The laryngeal mask airway device according to any one of Inventive Concepts 1-12, wherein the laryngeal mask airway device is configured such that when the inflatable balloon is disposed at the insertion location and the proximal inflatable pharyngeal portion of the inflatable balloon is inflated at the pressure of 25 cm H2O, the inflatable annular cuff spans both (a) a vertical space between a tongue and a pharyngeal back wall of the throat and (b) a lateral space between left and right tonsils, thereby forming a proximal seal between the oral cavity and a pharyngeal space distal to the proximal inflatable pharyngeal portion of the inflatable balloon.
Inventive Concept 31. The laryngeal mask airway device according to any one of Inventive Concepts 1-12,

wherein the laryngeal mask airway device is configured such that when a volume of 5 cc of ambient air, the volume measured at standard atmospheric pressure, is introduced into the inflation tube while the inflatable balloon is unconstrained, disposed in ambient air that is at standard atmospheric pressure, and is at an inflation pressure of 30 cm H2O:
the inflation pressure in the inflatable balloon, measured one minute after introduction of the volume of 5 cc of the ambient air, is less than 40 cm H2O.
   Inventive Concept 32. A laryngeal mask airway system including the laryngeal mask airway device according to any one of Inventive Concepts 1-12, the laryngeal mask airway system further including an insertion posterior protector, which includes an elongate sheet of material that is shaped so as to define:

a proximal coupling portion, which is shaped so as to define one or more couplers that are configured to removably couple the proximal coupling portion to the shaft proximally to the inflatable pharyngeal portion of the inflatable balloon, and

a distal protection portion, which is shaped so as to partially cover a posterior external surface of the inflatable balloon when the proximal coupling portion is removably coupled to the shaft, so as to protect the inflatable balloon from puncture during insertion thereof into the patient's oral cavity.

Inventive Concept 33. The laryngeal mask airway system according to Inventive Concept 32, wherein the one or more couplers are shaped so as to form snap-on couplings to the shaft.
Inventive Concept 34. The laryngeal mask airway system according to Inventive Concept 32, wherein the elongate sheet of material of the insertion posterior protector is further shaped so as to define a proximal handle, which is shaped so as to be disposed outside the patient's oral cavity when the inflatable balloon is disposed at the insertion location.

There is further provided, in accordance with an Inventive Concept 35 of the present invention, a laryngeal mask airway system including:

laryngeal mask airway device, which includes:
an inflatable balloon, which is shaped so as to define at least an inflatable annular cuff, which is insertable through an anterior opening of an oral cavity of a patient, the inflatable annular cuff shaped so as to define a cuff anterior side that is configured to stabilize the inflatable annular cuff with respect to a laryngeal inlet of the patient when the inflatable balloon is disposed at an insertion location within a throat of the patient;
a shaft having (a) a proximal end that is configured to be disposed outside the patient's oral cavity when the inflatable balloon is disposed at the insertion location, and (b) a distal end portion that is fixed to the inflatable balloon;
an airway tube, which passes through at least a longitudinal portion of the shaft, and has (a) a proximal end that is configured to be disposed outside the patient's oral cavity when the inflatable balloon is disposed at the insertion location, and (b) a distal end that is open through the inflatable annular cuff; and
an inflation tube having (a) a proximal end that is configured to be disposed outside the patient's oral cavity when the inflatable balloon is disposed at the insertion location, and (b) a distal end that is coupled in fluid communication with an inflatable chamber defined by the inflatable balloon, for supplying air to the inflatable chamber; and

an insertion posterior protector, which includes an elongate sheet of material that is shaped so as to define:
a proximal coupling portion, which is shaped so as to define one or more couplers that are configured to removably couple the proximal coupling portion to the shaft proximally to the inflatable balloon, and
a distal protection portion, which is shaped so as to partially cover a posterior external surface of the inflatable balloon when the proximal coupling portion is removably coupled to the shaft, so as to protect the inflatable balloon from puncture during insertion thereof into the patient's oral cavity.
   Inventive Concept 36. The laryngeal mask airway system according to Inventive Concept 35, wherein the one or more couplers are shaped so as to form snap-on couplings to the shaft.
   Inventive Concept 37. The laryngeal mask airway system according to Inventive Concept 35, wherein the elongate sheet of material of the insertion posterior protector is further shaped so as to define a proximal handle, which is shaped so as to be disposed outside the patient's oral cavity when the inflatable balloon is disposed at the insertion location.
   Inventive Concept 38. The laryngeal mask airway system according to Inventive Concept 35,

wherein the inflatable annular cuff is shaped so as to define a distal tip portion that includes a distal end of the inflatable annular cuff,

wherein the distal tip portion is configured to form a seal against an upper esophageal sphincter when the inflatable balloon is disposed at the insertion location, and

wherein a distal wall of the distal tip portion has a thickness of at least 2 mm.

Inventive Concept 39. The laryngeal mask airway device according to Inventive Concept 38, further including a drainage tube that (a) extends from a gastric channel having a distal gastric opening through the distal tip portion, and (b) has a proximal drainage port at location outside the patient's oral cavity when the inflatable balloon is disposed at the insertion location.
Inventive Concept 40. The laryngeal mask airway system according to Inventive Concept 35, wherein the cuff anterior side is configured to form a seal around the laryngeal inlet of the patient when the inflatable balloon is disposed at the insertion location.
Inventive Concept 41. The laryngeal mask airway system according to any one of Inventive Concepts 35-40,

wherein the inflatable balloon is shaped so as to define at least the inflatable annular cuff and a proximal inflatable pharyngeal portion, which are insertable through the anterior opening of the oral cavity,

wherein the proximal inflatable pharyngeal portion of the inflatable balloon forms an air-tight seal with an external surface of the shaft.

Inventive Concept 42. The laryngeal mask airway system according to Inventive Concept 41, wherein the inflatable balloon is configured such that when the inflatable chamber is filled with air at a pressure of 10 cm H2O and unconstrained, a volume of the air in a proximal one-third of the inflatable chamber equals at least 200% of a volume of the air in a distal one-third of the inflatable chamber,

the proximal one-third of the inflatable chamber extending distally from a proximal end of the inflatable chamber along one third of a length of the inflatable chamber,

the distal one-third of the inflatable chamber extending proximally from a distal end of the inflatable chamber along one third of the length of the inflatable chamber, and

the length measured along a straight line segment connecting (a) a distal centroid of the distal end of the inflatable chamber and (b) a proximal centroid of the proximal end of the inflatable chamber.

Inventive Concept 43. The laryngeal mask airway device according to Inventive Concept 42, wherein the proximal inflatable pharyngeal portion extends in both lateral directions away from the straight line segment.
Inventive Concept 44. The laryngeal mask airway device according to Inventive Concept 43, wherein the proximal inflatable pharyngeal portion is reflectionally symmetric about a plane of symmetry that extends in an anterior/posterior direction, wherein the straight line segment falls in the plane of symmetry.
Inventive Concept 45. The laryngeal mask airway system according to Inventive Concept 41, wherein the inflatable balloon is configured such that the proximal inflatable pharyngeal portion of the inflatable balloon forms the air-tight seal entirely around a longitudinal portion of the shaft.
Inventive Concept 46. The laryngeal mask airway system according to Inventive Concept 41, wherein the laryngeal mask airway device is configured such that when the inflatable chamber is filled with air at a pressure of 10 cm H2O and unconstrained, a portion of the proximal inflatable pharyngeal portion of the inflatable balloon bulges posteriorly with respect to the external surface of the shaft.
Inventive Concept 47. The laryngeal mask airway system according to Inventive Concept 41, wherein a distal portion of the shaft passes through at least a portion of the proximal inflatable pharyngeal portion of the inflatable balloon, such that, when the inflatable chamber is filled with air at a pressure of 10 cm H2O and unconstrained, a portion of the inflatable chamber defined by the proximal inflatable pharyngeal portion surrounds 360 degrees of the distal portion of the shaft.

There is still further provided, in accordance with an Inventive Concept 48 of the present invention, a laryngeal mask airway device including:

an inflatable balloon, which is (a) shaped so as to define at least an inflatable annular cuff, and (b) insertable through an anterior opening of an oral cavity of a patient, the inflatable annular cuff shaped so as to define a cuff anterior side that is configured to surround a laryngeal inlet of the patient when the inflatable annular cuff is disposed at an insertion location within a throat of the patient, wherein the cuff anterior side, along a proximal side of the inflatable annular cuff, is shaped so as to define a posteriorly-recessed portion;

an airway tube, which extends proximally from the inflatable balloon, and which has (a) a proximal end that is configured to be disposed outside the patient's oral cavity when the inflatable annular cuff is disposed at the insertion location, and (b) a distal end that is open through the inflatable annular cuff; and

an inflation tube having (a) a proximal end that is configured to be disposed outside the patient's oral cavity when the inflatable annular cuff is disposed at the insertion location, and (b) a distal end that is coupled in fluid communication with an inflatable chamber of the inflatable balloon, for supplying air to the inflatable chamber.

Inventive Concept 49. The laryngeal mask airway device according to Inventive Concept 48, wherein the posteriorly-recessed portion of the cuff anterior side is curved.
Inventive Concept 50. The laryngeal mask airway device according to Inventive Concept 48, wherein the anterior side of the laryngeal mask airway device is configured to form a seal around the laryngeal inlet of the patient when the inflatable annular cuff is disposed at the insertion location.

There is additionally provided, in accordance with an Inventive Concept 51 of the present invention, a laryngeal mask airway device including:

an inflatable balloon, which is insertable through an anterior opening of an oral cavity of a patient;

an airway tube, which (i) is coupled to the inflatable balloon, and (ii) has a proximal end that is configured to be disposed outside the patient's oral cavity when the inflatable balloon is disposed at an insertion location within a throat of the patient, wherein an anterior side of the laryngeal mask airway device is configured to stabilize the laryngeal mask airway device with respect to a laryngeal inlet of the patient when the inflatable balloon is disposed at the insertion location; and

an inflation tube (i) having (a) a proximal end that is configured to be disposed outside the patient's oral cavity when the inflatable balloon is disposed at the insertion location, and (b) a distal end that is coupled in fluid communication with an inflatable chamber defined by the inflatable balloon, for supplying air to the inflatable balloon, and (ii) including an inflation valve that is configured to be disposed outside the patient's oral cavity when the inflatable balloon is disposed at the insertion location,

wherein the laryngeal mask airway device is configured such that when the inflatable balloon, while the inflation valve is closed and a pressure within the inflatable balloon equals ambient pressure, is inserted from outside the oral cavity of the patient to the insertion location within the throat without opening the inflation valve, the pressure within the inflatable balloon increases from the ambient pressure to an inserted-location pressure, the inserted-location pressure less than 60 cm H2O.

Inventive Concept 52. The laryngeal mask airway device according to Inventive Concept 51, wherein the inserted-location pressure is less than 45 cm H2O.
Inventive Concept 53. The laryngeal mask airway device according to Inventive Concept 52, wherein the inserted-location pressure is less than 30 cm H2O.
Inventive Concept 54. The laryngeal mask airway device according to Inventive Concept 51, wherein the anterior side of the laryngeal mask airway device is configured to form a seal around the laryngeal inlet of the patient when the inflatable balloon is disposed at the insertion location.
Inventive Concept 55. The laryngeal mask airway device according to Inventive Concept 51,

wherein the inflatable balloon is shaped so as to define at least an inflatable annular cuff and a proximal inflatable pharyngeal portion, the inflatable annular cuff shaped so as to define a cuff anterior side that is configured to stabilize the inflatable annular cuff with respect to the laryngeal inlet of the patient when the inflatable balloon is disposed at the insertion location,

wherein the laryngeal mask airway device further includes a shaft having (a) a proximal end that is configured to be disposed outside the patient's oral cavity when the inflatable balloon is disposed at the insertion location, and (b) a distal end portion that is fixed to the inflatable balloon,

wherein the airway tube passes through at least a longitudinal portion of the shaft,

wherein the proximal inflatable pharyngeal portion of the inflatable balloon forms an air-tight seal with an external surface of the shaft, and

wherein the inflatable balloon is configured such that when the inflatable chamber is filled with air at a pressure of 10 cm H2O and unconstrained, a volume of the air in a proximal one-third of the inflatable chamber equals at least 200% of a volume of the air in a distal one-third of the inflatable chamber,
the proximal one-third of the inflatable chamber extending distally from a proximal end of the inflatable chamber along one third of a length of the inflatable chamber,
the distal one-third of the inflatable chamber extending proximally from a distal end of the inflatable chamber along one third of the length of the inflatable chamber, and
the length measured along a straight line segment connecting (a) a distal centroid of the distal end of the inflatable chamber and (b) a proximal centroid of the proximal end of the inflatable chamber.
   Inventive Concept 56. The laryngeal mask airway device according to Inventive Concept 51,

wherein the laryngeal mask airway device is configured such that when a volume of 5 cc of ambient air, the volume measured at standard atmospheric pressure, is introduced into the inflation tube while the inflatable balloon is unconstrained, disposed in ambient air that is at standard atmospheric pressure, and is at an inflation pressure of 30 cm H2O:
the inflation pressure in the inflatable balloon, measured one minute after introduction of the volume of 5 cc of the ambient air, is less than 40 cm H2O.

There is yet additionally provided, in accordance with an Inventive Concept 57 of the present invention, a laryngeal mask airway device including:

an inflatable balloon, which is (a) shaped so as to define at least an inflatable annular cuff, and (b) insertable through an anterior opening of an oral cavity of a patient, the inflatable annular cuff shaped so as to define:
a cuff anterior side that is configured to stabilize the inflatable annular cuff with respect to a laryngeal inlet of the patient when the inflatable balloon is disposed at an insertion location within a throat of the patient, and
a distal tip portion, which (a) includes a distal end of the inflatable annular cuff, and (b) is configured to form a seal against an upper esophageal sphincter when the inflatable balloon is disposed at the insertion location, wherein a distal wall of the distal tip portion has a thickness of at least 2 mm;

an airway tube having (a) a proximal end that is configured to be disposed outside the patient's oral cavity when the inflatable balloon is disposed at the insertion location, and (b) a distal end that is open through the inflatable annular cuff; and

an inflation tube having (a) a proximal end that is configured to be disposed outside the patient's oral cavity when the inflatable balloon is disposed at the insertion location, and (b) a distal end that is coupled in fluid communication with an inflatable chamber of the inflatable balloon, for supplying air to the inflatable chamber.

Inventive Concept 58. The laryngeal mask airway device according to Inventive Concept 57, further including a drainage tube that (a) extends from a gastric channel having a distal gastric opening through the distal tip portion, and (b) has a proximal drainage port at location outside the patient's oral cavity when the inflatable balloon is disposed at the insertion location.

There is also provided, in accordance with an Inventive Concept 59 of the present invention, a method including:

inserting an inflatable balloon of a laryngeal mask airway device through an anterior opening of an oral cavity of a patient, the inflatable balloon shaped so as to define at least an inflatable annular cuff and a proximal inflatable pharyngeal portion, and disposing the inflatable balloon at an insertion location within a throat of the patient, such that (a) a proximal end of an inflation tube is disposed outside the patient's oral cavity, wherein a distal end of the inflation tube is coupled in fluid communication with an inflatable chamber defined by the inflatable balloon, for supplying air to the inflatable balloon, (b) a proximal end of a shaft of the laryngeal mask airway device is disposed outside the patient's oral cavity, wherein a distal end portion of the shaft is fixed to the inflatable balloon, (c) a proximal end of an airway tube of the laryngeal mask airway device is disposed outside the patient's oral cavity, the airway tube passing through at least a longitudinal portion of the shaft, and having a distal end that is open through the inflatable annular cuff, wherein the proximal inflatable pharyngeal portion of the inflatable balloon forms an air-tight seal with an external surface of the shaft, and (d) a cuff anterior side of the inflatable annular cuff stabilizes the inflatable annular cuff with respect to a laryngeal inlet of the patient; and

ventilating lungs of the patient using the laryngeal mask airway device,

wherein the inflatable balloon is configured such that when the inflatable chamber is filled with air at a pressure of 10 cm H2O and unconstrained, a volume of the air in a proximal one-third of the inflatable chamber equals at least 200% of a volume of the air in a distal one-third of the inflatable chamber,
the proximal one-third of the inflatable chamber extending distally from a proximal end of the inflatable chamber along one third of a length of the inflatable chamber,
the distal one-third of the inflatable chamber extending proximally from a distal end of the inflatable chamber along one third of the length of the inflatable chamber, and
the length measured along a straight line segment connecting (a) a distal centroid of the distal end of the inflatable chamber and (b) a proximal centroid of the proximal end of the inflatable chamber.

There is further provided, in accordance with an Inventive Concept 60 of the present invention, a method including:

inserting an inflatable balloon of a laryngeal mask airway device through an anterior opening of an oral cavity of a patient, the inflatable balloon shaped so as to define at least an inflatable annular cuff, and disposing the inflatable balloon at an insertion location within a throat of the patient, such that (a) a proximal end of an inflation tube is disposed outside the patient's oral cavity, wherein a distal end of the inflation tube is coupled in fluid communication with an inflatable chamber defined by the inflatable balloon, for supplying air to the inflatable balloon, (b) a proximal end of a shaft of the laryngeal mask airway device is disposed outside the patient's oral cavity, wherein a distal end portion of the shaft is fixed to the inflatable balloon, (c) a proximal end of an airway tube of the laryngeal mask airway device is disposed outside the patient's oral cavity, the airway tube passing through at least a longitudinal portion of the shaft, and having a distal end that is open through the inflatable annular cuff, and (d) a cuff anterior side of the inflatable annular cuff stabilizes the inflatable annular cuff with respect to a laryngeal inlet of the patient; and

ventilating lungs of the patient using the laryngeal mask airway device,

wherein inserting the inflatable balloon and disposing the inflatable balloon at the insertion location includes inserting the inflatable balloon and disposing the inflatable balloon at the insertion location while:
a proximal coupling portion of an elongate sheet of material of an insertion posterior protector is removably coupled to the shaft proximally to the inflatable balloon by one or more couplers of the proximal coupling portion, and
a distal protection portion of the elongate sheet of material partially covers a posterior external surface of the inflatable balloon, so as to protect the inflatable balloon from puncture during insertion thereof into the patient's oral cavity, and

wherein the method further includes, after inserting the inflatable balloon and disposing the inflatable balloon at the insertion location, decoupling the insertion posterior protector from the shaft and removing the insertion posterior protector from the patient's oral cavity while the inflatable balloon remains disposed at the insertion location.

Inventive Concept 61. The method according to Inventive Concept 60, wherein the one or more couplers are shaped so as to form snap-on couplings to the shaft.
Inventive Concept 62. The method according to Inventive Concept 60, wherein inserting the inflatable balloon and disposing the inflatable balloon at the insertion location includes inserting the inflatable balloon and disposing the inflatable balloon at the insertion location using a proximal handle of the elongate sheet of material, while the proximal handle is disposed outside the patient's oral cavity.

There is still further provided, in accordance with an Inventive Concept 63 of the present invention, a method including:

inserting an inflatable balloon of a laryngeal mask airway device through an anterior opening of an oral cavity of a patient, the inflatable balloon shaped so as to define at least an inflatable annular cuff, and disposing the inflatable balloon at an insertion location within a throat of the patient, such that (a) a proximal end of an inflation tube is disposed outside the patient's oral cavity, wherein a distal end of the inflation tube is coupled in fluid communication with an inflatable chamber defined by the inflatable balloon, for supplying air to the inflatable balloon, (b) a proximal end of an airway tube of the laryngeal mask airway device is disposed outside the patient's oral cavity, wherein the airway tube has a distal end that is open through the inflatable annular cuff, and (c) a cuff anterior side of the inflatable annular cuff surrounds a laryngeal inlet of the patient; and

ventilating lungs of the patient using the laryngeal mask airway device,

wherein the cuff anterior side, along a proximal side of the inflatable annular cuff, is shaped so as to define a posteriorly-recessed portion.

There is additionally provided, in accordance with an Inventive Concept 64 of the present invention, a method including:

inserting an inflatable balloon of a laryngeal mask airway device through an anterior opening of an oral cavity of a patient and disposing the inflatable balloon at an insertion location within a throat of the patient, such that (a) a proximal end of an inflation tube is disposed outside the patient's oral cavity, wherein a distal end of the inflation tube is coupled in fluid communication with an inflatable chamber defined by the inflatable balloon, for supplying air to the inflatable balloon, (b) an inflation valve of the inflation tube and a proximal end of an airway tube of the laryngeal mask airway device are disposed outside the patient's oral cavity, the airway tube coupled to the inflatable balloon, and (c) an anterior side of the laryngeal mask airway device stabilizes the laryngeal mask airway device with respect to a laryngeal inlet of the patient; and

ventilating lungs of the patient using the laryngeal mask airway device,

wherein inserting the inflatable balloon includes inserting the inflatable balloon, while the inflation valve is closed, from outside the oral cavity of the patient to the insertion location within the throat without opening the inflation valve, and

wherein the laryngeal mask airway device is configured such that when the inflatable balloon, while the inflation valve is closed and a pressure within the inflatable balloon equals ambient pressure, is inserted from outside the oral cavity of the patient to the insertion location within the throat without opening the inflation valve, the pressure within the inflatable balloon increases from the ambient pressure to an inserted-location pressure, the inserted-location pressure less than 60 cm H2O.

Inventive Concept 65. The method according to Inventive Concept 64, wherein inserting the inflatable balloon includes inserting the inflatable balloon such that when the inflatable balloon is disposed at the insertion location, a proximal inflatable pharyngeal portion of the inflatable balloon spans both (a) a vertical space between a tongue and a pharyngeal back wall of the throat and (b) a lateral space between left and right tonsils, thereby forming a proximal seal between the oral cavity and a pharyngeal space distal to the proximal inflatable pharyngeal portion of the inflatable balloon.
Inventive Concept 66. The method according to Inventive Concept 64, wherein inserting the inflatable balloon while the inflation valve is closed includes inserting the inflatable balloon while the inflation valve is closed and the pressure within the inflatable balloon equals ambient pressure.
Inventive Concept 67. The method according to Inventive Concept 64, wherein inserting the inflatable balloon while the inflation valve is closed includes inserting the inflatable balloon while the inflation valve is closed without deflating the inflatable balloon before inserting the inflatable balloon.
Inventive Concept 68. The method according to Inventive Concept 64, wherein the method does not include measuring the pressure within the inflatable balloon.
Inventive Concept 69. The method according to Inventive Concept 64, wherein the method does not include adding air to or removing air from the inflatable balloon after inserting the inflatable balloon to the insertion location.
Inventive Concept 70. The method according to Inventive Concept 64, wherein the method does not include adjusting the pressure within the inflatable balloon after inserting the inflatable balloon to the insertion location.
Inventive Concept 71. The method according to Inventive Concept 64, wherein the inserted-location pressure is less than 45 cm H2O.
Inventive Concept 72. The method according to Inventive Concept 71, wherein the inserted-location pressure is less than 30 cm H2O.
Inventive Concept 73. The method according to Inventive Concept 64, wherein the anterior side of the laryngeal mask airway device is configured to form a seal around the laryngeal inlet of the patient when the inflatable balloon is disposed at the insertion location.
Inventive Concept 74. The method according to Inventive Concept 64,

wherein the inflatable balloon is shaped so as to define at least an inflatable annular cuff and a proximal inflatable pharyngeal portion, the inflatable annular cuff shaped so as to define a cuff anterior side that is configured to stabilize the inflatable annular cuff with respect to the laryngeal inlet of the patient when the inflatable balloon is disposed at the insertion location,

wherein the laryngeal mask airway device further includes a shaft having (a) a proximal end that is configured to be disposed outside the patient's oral cavity when the inflatable balloon is disposed at the insertion location, and (b) a distal end portion that is fixed to the inflatable balloon,

wherein the airway tube passes through at least a longitudinal portion of the shaft,

wherein the proximal inflatable pharyngeal portion of the inflatable balloon forms an air-tight seal with an external surface of the shaft, and

wherein the inflatable balloon is configured such that when the inflatable chamber is filled with air at a pressure of 10 cm H2O and unconstrained, a volume of the air in a proximal one-third of the inflatable chamber equals at least 200% of a volume of the air in a distal one-third of the inflatable chamber,
the proximal one-third of the inflatable chamber extending distally from a proximal end of the inflatable chamber along one third of a length of the inflatable chamber,
the distal one-third of the inflatable chamber extending proximally from a distal end of the inflatable chamber along one third of the length of the inflatable chamber, and
the length measured along a straight line segment connecting (a) a distal centroid of the distal end of the inflatable chamber and (b) a proximal centroid of the proximal end of the inflatable chamber.

There is yet additionally provided, in accordance with an Inventive Concept 75 of the present invention, a method including:

inserting an inflatable balloon of a laryngeal mask airway device through an anterior opening of an oral cavity of a patient, the inflatable balloon shaped so as to define at least an inflatable annular cuff, and disposing the inflatable balloon at an insertion location within a throat of the patient, such that (a) a proximal end of an inflation tube is disposed outside the patient's oral cavity, wherein a distal end of the inflation tube is coupled in fluid communication with an inflatable chamber defined by the inflatable balloon, for supplying air to the inflatable balloon, (b) a proximal end of an airway tube of the laryngeal mask airway device is disposed outside the patient's oral cavity, the airway tube having a distal end that is open through the inflatable annular cuff, (c) a cuff anterior side of the inflatable annular cuff stabilizes the inflatable annular cuff with respect to a laryngeal inlet of the patient, and (d) a distal tip portion, which includes a distal end of the inflatable annular cuff, forms a seal against an upper esophageal sphincter, wherein a distal wall of the distal tip portion has a thickness of at least 2 mm; and

ventilating lungs of the patient using the laryngeal mask airway device.

Inventive Concept 76. The method according to Inventive Concept 75, wherein the laryngeal mask airway device further includes a drainage tube that (a) extends from a gastric channel having a distal gastric opening through the distal tip portion, and (b) has a proximal drainage port at location outside the patient's oral cavity when the inflatable balloon is disposed at the insertion location.

There is also provided, in accordance with an Inventive Concept 77 of the present invention, a method of manufacturing a laryngeal mask airway device, the method including:

attaching an inflatable balloon to a shaft while the inflatable balloon is inverted, by sealing at least a portion of a perimeter of a lateral opening of the inverted inflatable balloon to the shaft, such that the inverted inflatable balloon extends anteriorly from the shaft; and

thereafter, inverting the inverted inflatable balloon such that a posterior portion of the inflatable balloon covers at least a portion of a posterior region of the shaft,

wherein the inflatable balloon is configured to be inserted through an anterior opening of an oral cavity of a patient and disposed at an insertion location within a throat of the patient, and wherein the shaft has a proximal end that is configured to be disposed outside the patient's oral cavity when the inflatable balloon is disposed at the insertion location within the throat.

Inventive Concept 78. The method according to Inventive Concept 77, further including, after inverting the inverted inflatable balloon, sealing the inflatable balloon with an air-tight seal with an external surface of the shaft.
Inventive Concept 79. The method according to Inventive Concept 77,

wherein the inverted inflatable balloon is shaped so as to define a proximal end opening, which, after the inverted inflatable balloon is attached to the shaft and before the inflatable balloon is inverted, is not in direct contact with any portions of the laryngeal mask airway device other than the inverted inflatable balloon,

wherein inverting the inverted inflatable balloon includes passing, in a distal-to-proximal direction, the proximal end opening of the inflatable balloon over and around a distal end opening of the shaft, until the proximal end opening of the inflatable balloon surrounds the shaft, and

wherein the method further includes, after inverting the inverted inflatable balloon, sealing the proximal end opening of the inflatable balloon to an external surface of the shaft.

There is further provided, in accordance with an Inventive Concept 80 of the present invention, a laryngeal mask airway device prepared by a process including the steps of:

attaching an inflatable balloon to a shaft while the inflatable balloon is inverted, by sealing at least a portion of a perimeter of a lateral opening of the inverted inflatable balloon to the shaft, such that the inverted inflatable balloon extends anteriorly from the shaft; and

thereafter, inverting the inverted inflatable balloon such that a posterior portion of the inflatable balloon covers at least a portion of a posterior region of the shaft,

wherein the inflatable balloon is configured to be inserted through an anterior opening of an oral cavity of a patient and disposed at an insertion location within a throat of the patient, and wherein the shaft has a proximal end that is configured to be disposed outside the patient's oral cavity when the inflatable balloon is disposed at the insertion location within the throat.

There is still further provided, in accordance with an Inventive Concept 81 of the present invention, a laryngeal mask airway device including:

an inflatable balloon, which is (a) shaped so as to define at least an inflatable annular cuff, and (b) insertable through an anterior opening of an oral cavity of a patient, the inflatable annular cuff shaped so as to define a cuff anterior side that is configured to stabilize the inflatable annular cuff with respect to a laryngeal inlet of the patient when the inflatable balloon is disposed at an insertion location within a throat of the patient;

a shaft having (a) a proximal end that is configured to be disposed outside the patient's oral cavity when the inflatable balloon is disposed at the insertion location, and (b) a distal end portion that is fixed to the inflatable balloon;

an airway tube, which passes through at least a longitudinal portion of the shaft, and has (a) a proximal end that is configured to be disposed outside the patient's oral cavity when the inflatable balloon is disposed at the insertion location, and (b) a distal end that is open through the inflatable annular cuff;

an inflation tube having (a) a proximal end that is configured to be disposed outside the patient's oral cavity when the inflatable balloon is disposed at the insertion location, and (b) a distal end that is coupled in fluid communication with an inflatable chamber defined by the inflatable balloon, for supplying air to the inflatable chamber;

a distal tip portion, which is located distal to the inflatable annular cuff, and which is configured to form a seal against an upper esophageal sphincter when the inflatable balloon is disposed at the insertion location; and

an inner support, which is mostly covered by the inflatable balloon,

wherein the inflatable balloon is shaped so as to define a distal inflatable tip portion, which is in fluid communication with the rest of the inflatable chamber of the inflatable balloon, and

wherein the distal inflatable tip portion entirely surrounds a longitudinal portion of the distal tip portion.

Inventive Concept 82. The laryngeal mask airway device according to Inventive Concept 81, wherein a distal end of the longitudinal portion is at a distance from a distal-most tip of the distal tip portion, the distance between 1 and 5 mm.
Inventive Concept 83. The laryngeal mask airway device according to Inventive Concept 81, wherein the longitudinal portion has a length of between 1 and 3 cm.
Inventive Concept 84. The laryngeal mask airway device according to Inventive Concept 83, wherein the length is between 2 and 4 cm.
Inventive Concept 85. The laryngeal mask airway device according to Inventive Concept 84, wherein the length is between 1 and 3 cm.
Inventive Concept 86. The laryngeal mask airway device according to Inventive Concept 81, wherein the longitudinal portion has a greatest width, measured from side to side of the inflatable balloon, of no more than 20 mm along a length of the longitudinal portion.
Inventive Concept 87. The laryngeal mask airway device according to Inventive Concept 81, wherein the distal tip portion includes a distal-most non-balloon-covered tip portion, defined by the inner support.
Inventive Concept 88. The laryngeal mask airway device according to Inventive Concept 81, wherein a proximal end of the distal inflatable tip portion is at least partially coupled to the inner support, so as to restrict air flow between the distal inflatable tip portion and the inflatable annular cuff, without entirely blocking the air flow.
Inventive Concept 89. The laryngeal mask airway device according to Inventive Concept 81, further including a drainage tube that:

extends from a gastric channel that is defined by the inner support,

has a distal gastric opening through the distal tip portion, and

has a proximal drainage port at location outside the patient's oral cavity when the inflatable balloon is disposed at the insertion location.

Inventive Concept 90. The laryngeal mask airway device according to any one of Inventive Concepts 81-89, wherein the inflatable balloon is shaped so as to further define a proximal inflatable pharyngeal portion, which forms an air-tight seal with an external surface of the shaft.
Inventive Concept 91. The laryngeal mask airway device according to any one of Inventive Concepts 81-89,

wherein the laryngeal mask airway device is configured such that when a volume of 5 cc of ambient air, the volume measured at standard atmospheric pressure, is introduced into the inflation tube while the inflatable balloon is unconstrained, disposed in ambient air that is at standard atmospheric pressure, and is at an inflation pressure of 30 cm H2O:
the inflation pressure in the inflatable balloon, measured one minute after introduction of the volume of 5 cc of the ambient air, is less than 40 cm H2O.

There is additionally provided, in accordance with an Inventive Concept 92 of the present invention, a laryngeal mask airway device including:

an inflatable annular cuff, which is insertable through an anterior opening of an oral cavity of a patient and is shaped so as to define:
a cuff anterior side that is configured to stabilize the inflatable annular cuff with respect to a laryngeal inlet of the patient when the inflatable annular cuff is disposed at an insertion location within a throat of the patient, and is shaped so at to define and surround a ventilation port, and
a distal tip that has a length, measured between a distal end of the distal tip and a proximal straight border of the distal tip, of at least 25 mm, when the inflatable annular cuff is filled with air at a pressure of 5 cm H2O and unconstrained, such that the distal tip forms a seal within an upper part of an esophagus of the patient when the inflatable annular cuff is disposed at the insertion location,
wherein the proximal straight border of the distal tip is located at a longitudinal location along the inflatable annular cuff at which the inflatable annular cuff has a width of 29 mm, measured from side to side of the inflatable annular cuff;

a shaft having (a) a proximal end that is configured to be disposed outside the patient's oral cavity when the inflatable annular cuff is disposed at the insertion location, and (b) a distal end portion that is fixed to the inflatable annular cuff;

an airway tube, which passes through at least a longitudinal portion of the shaft, and has (a) a proximal end that is configured to be disposed outside the patient's oral cavity when the inflatable annular cuff is disposed at the insertion location, and (b) a distal end that is in fluid communication with the ventilation port; and

an inflation tube having (a) a proximal end that is configured to be disposed outside the patient's oral cavity when the inflatable annular cuff is disposed at the insertion location, and (b) a distal end that is coupled in fluid communication with an interior of the inflatable annular cuff.

Inventive Concept 93. The laryngeal mask airway device according to Inventive Concept 92, wherein the length of the distal tip is at least 30 mm, when the inflatable annular cuff is filled with the air at the pressure of 5 cm H2O and unconstrained.
Inventive Concept 94. The laryngeal mask airway device according to Inventive Concept 92, wherein the length of the distal tip is less than 40 mm, when the inflatable annular cuff is filled with the air at the pressure of 5 cm H2O and unconstrained.
Inventive Concept 95. The laryngeal mask airway device according to Inventive Concept 92, wherein the cuff anterior side is configured to form a seal around the laryngeal inlet of the patient when the inflatable annular cuff is disposed at the insertion location.
Inventive Concept 96. The laryngeal mask airway device according to Inventive Concept 92, wherein the distal tip is configured to form a seal against an upper esophageal sphincter when the inflatable annular cuff is disposed at the insertion location.
Inventive Concept 97. The laryngeal mask airway device according to any one of Inventive Concepts 92-96, further including a drainage tube that (a) extends from a gastric channel having a distal gastric opening through the distal tip, and (b) has a proximal drainage port at location outside the patient's oral cavity when the inflatable annular cuff is disposed at the insertion location.
Inventive Concept 98. The laryngeal mask airway device according to any one of Inventive Concepts 92-96,

wherein the laryngeal mask airway device is configured such that when a volume of 5 cc of ambient air, the volume measured at standard atmospheric pressure, is introduced into the inflation tube while the inflatable annular cuff is unconstrained, disposed in ambient air that is at standard atmospheric pressure, and is at an inflation pressure of 30 cm H2O:
the inflation pressure in the inflatable annular cuff, measured one minute after introduction of the volume of 5 cc of the ambient air, is less than 40 cm H2O.

There is yet additionally provided, in accordance with an Inventive Concept 99 of the present invention, a method including:

inserting an inflatable annular cuff of a laryngeal mask airway device through an anterior opening of an oral cavity of a patient, the inflatable annular cuff shaped so as to define (a) a cuff anterior side that is shaped so at to define and surround a ventilation port, and (b) a distal tip that has a length, measured between a distal end of the distal tip and a proximal straight border of the distal tip, of at least 25 mm, when the inflatable annular cuff is filled with air at a pressure of 5 cm H2O and unconstrained, wherein the proximal straight border of the distal tip is located at a longitudinal location along the inflatable annular cuff at which the inflatable annular cuff has a width of 29 mm, measured from side to side of the inflatable annular cuff;

disposing the inflatable annular cuff at an insertion location within a throat of the patient, such that:
(a) a proximal end of an inflation tube is disposed outside the patient's oral cavity, wherein a distal end of the inflation tube is coupled in fluid communication with an interior of the inflatable annular cuff,
(b) a proximal end of a shaft is disposed outside the patient's oral cavity, wherein a distal end portion of the shaft is fixed to the inflatable annular cuff,
(c) a proximal end of an airway tube of the laryngeal mask airway device is disposed outside the patient's oral cavity, the airway tube passing through at least a longitudinal portion of the shaft, and having a distal end that is in fluid communication with the ventilation port,
(d) the cuff anterior side stabilizes the inflatable annular cuff with respect to a laryngeal inlet of the patient, and
(e) the distal tip portion forms a seal within an upper part of an esophagus of the patient; and

ventilating lungs of the patient using the laryngeal mask airway device.

The present invention will be more fully understood from the following detailed description of embodiments thereof, taken together with the drawings, in which:

### BRIEF DESCRIPTION OF THE DRAWINGS

FIGS. 1A-H are schematic illustrations of several views of a laryngeal mask airway device, in accordance with an application of the present invention;

FIGS. 2A-D are schematic illustrations of several views of the laryngeal mask airway device of FIGS. 1A-H, in accordance with an application of the present invention;

FIGS. 3A-E are schematic cross-sectional views of the laryngeal mask airway device of FIGS. 1A-H, in accordance with an application of the present invention;

FIGS. 4A-B are schematic illustrations of an insertion posterior protector coupled to and decoupled from the laryngeal mask airway device of FIGS. 1A-H, respectively, in accordance with an application of the present invention;

FIG. 5 is a schematic illustration of the laryngeal mask airway device of FIG. 1A-H inserted through an anterior opening of an oral cavity of a patient, in accordance with an application of the present invention;

FIG. 6 is a graph showing pressures measured within prior art laryngeal mask airway cuffs and an inflatable balloon of the laryngeal mask airway device of FIGS. 1A-H, as measured during an experiment conducted by the inventor of the present invention;

FIGS. 7A-G are schematic illustrations of a method of manufacturing a laryngeal mask airway device, in accordance with an application of the present invention;

FIGS. 8A-C are schematic illustrations of several views of another laryngeal mask airway device, in accordance with an application of the present invention;

FIGS. 9A-B are schematic illustrations of several views of the laryngeal mask airway device of FIGS. 8A-C, in accordance with an application of the present invention;

FIGS. 10A-B are schematic cross-sectional views of the laryngeal mask airway device of FIGS. 8A-C, in accordance with an application of the present invention;

FIG. 11 is a schematic illustration of the insertion posterior protector of FIGS. 4A-B coupled to the laryngeal mask airway device of FIGS. 8A-C, in accordance with an application of the present invention;

FIG. 12 is a graph showing pressures measured within prior art laryngeal mask airway cuffs and an inflatable balloon of the laryngeal mask airway device of FIGS. 8A-C, as measured during an experiment conducted on behalf of the inventor of the present invention;

FIG. 13 is a schematic illustration of one stage of a method of manufacturing the laryngeal mask airway device of FIGS. 8A-C, in accordance with an application of the present invention;

FIGS. 14A-B are schematic illustrations of anterior and posterior sides, respectively, of a laryngeal mask airway device known in the art; the laryngeal mask airway device incudes a drainage tube having a distal gastric opening;

FIGS. 15A-B are schematic illustrations of a cuff anterior side and a cuff posterior side, respectively, of another laryngeal mask airway device, in accordance with an application of the present invention;

FIGS. 16A-B are schematic illustrations of anterior and posterior sides, respectively, of a laryngeal mask airway device known in the art, with an inflatable annular cuff thereof disposed at an insertion location within a throat of a patient;

FIGS. 17A-B are schematic illustrations of the cuff anterior and posterior sides, respectively, of the laryngeal mask airway device of FIGS. 15A-B, in accordance with an application of the present invention; and

FIG. 18 is a schematic illustration of the laryngeal mask airway device of FIGS. 15A-B disposed at an insertion location within the throat of a patient, in accordance with an application of the present invention.

### DETAILED DESCRIPTION OF APPLICATIONS

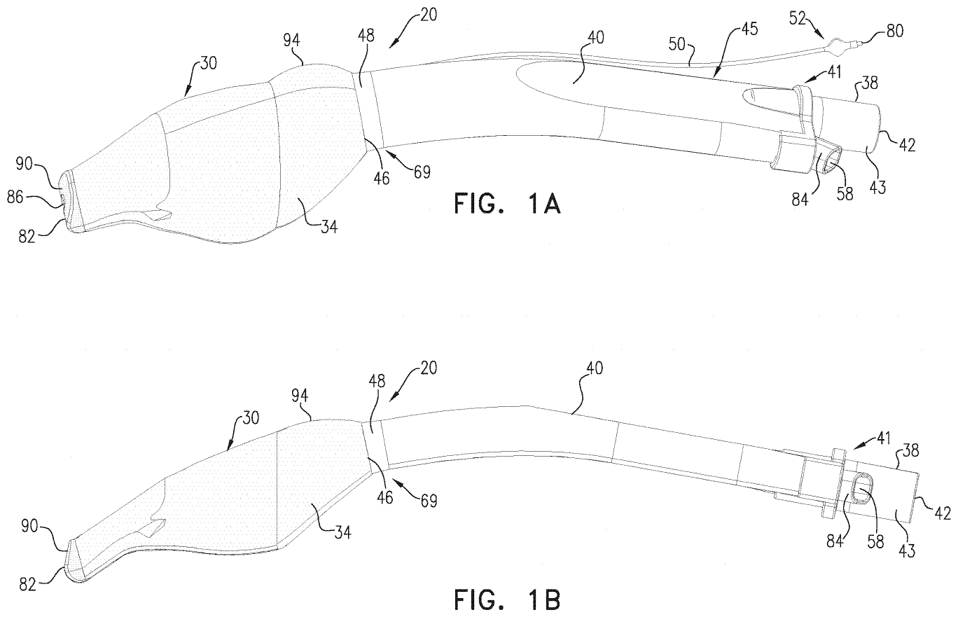

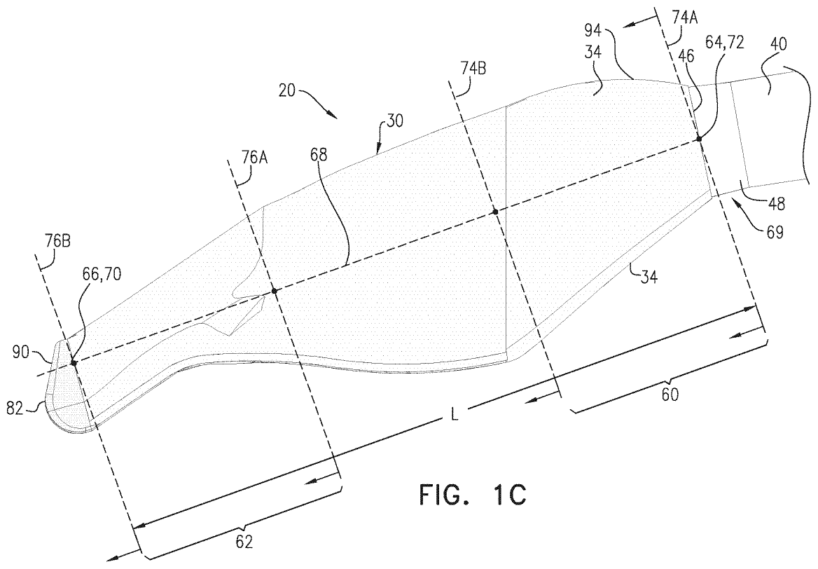

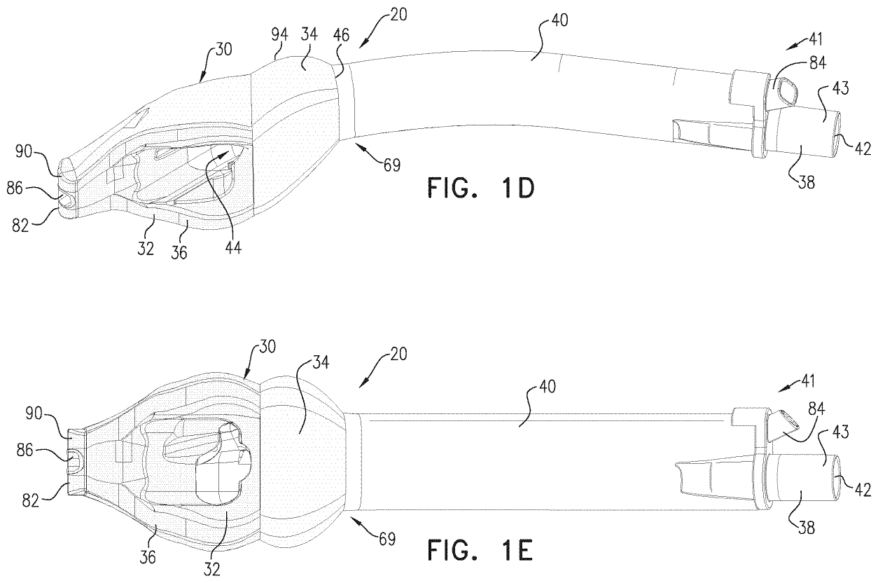

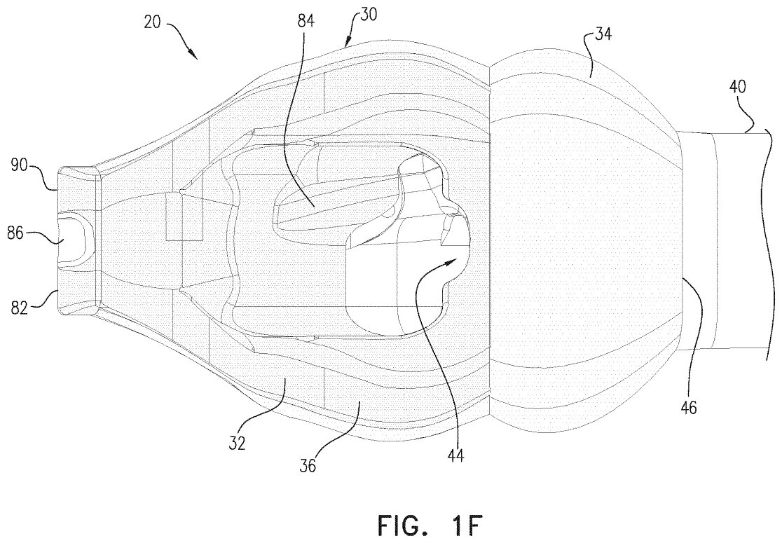

Reference is made to FIGS. 1A-H, which are schematic illustrations of several views of a laryngeal mask airway device 20, in accordance with an application of the present invention.

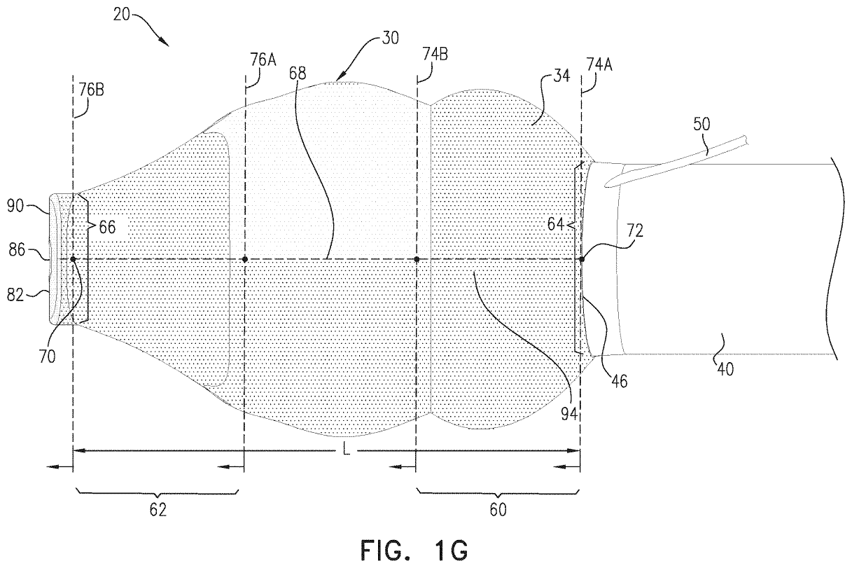

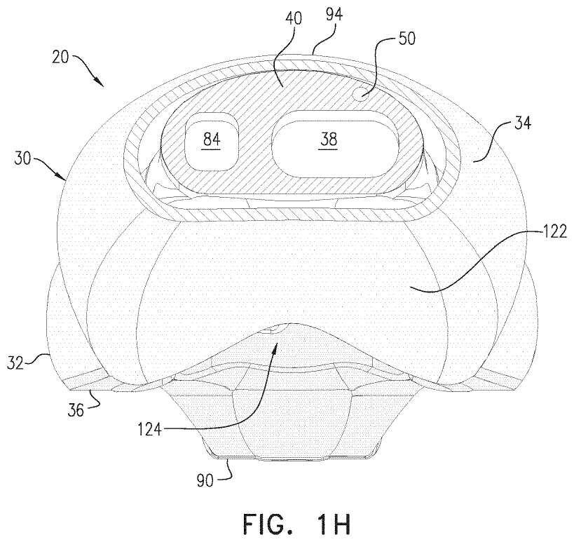

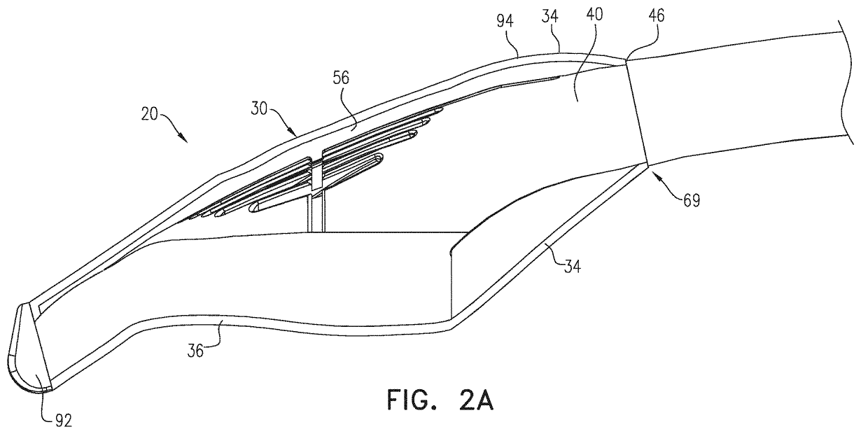

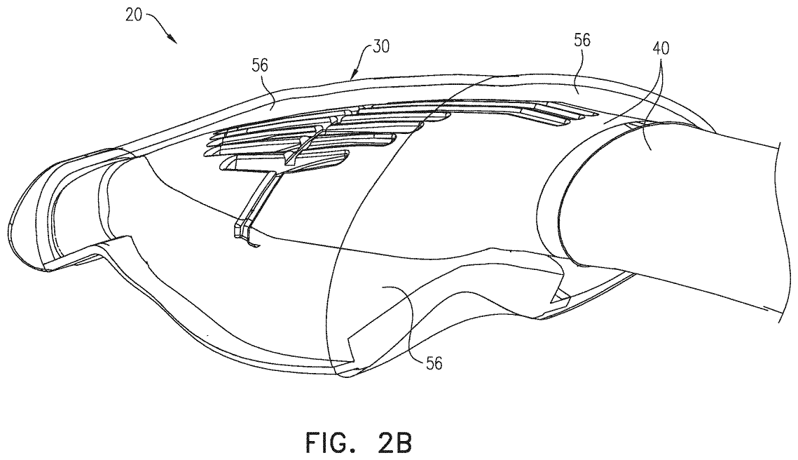

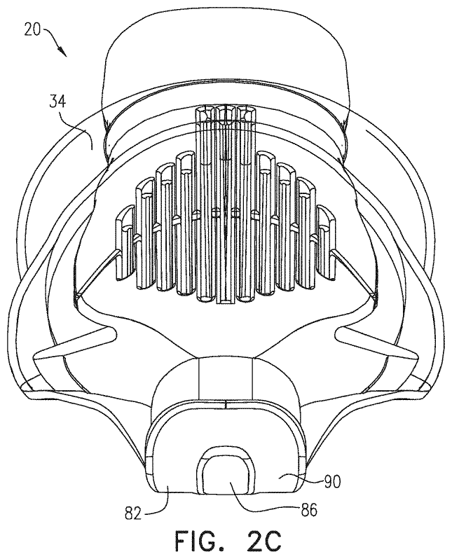

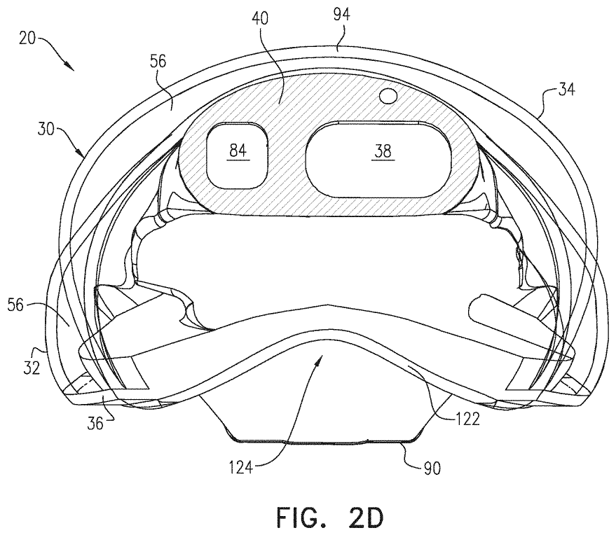

Reference is also made to FIGS. 2A-D, which are schematic illustrations of several views of laryngeal mask airway device 20, in accordance with an application of the present invention. In FIGS. 2A-D, a portion of an inflatable balloon 30 of laryngeal mask airway device 20 is shown as translucent to show some elements the view of which would otherwise be blocked by the portion of inflatable balloon 30. In practice, inflatable balloon 30 may be translucent, transparent, or opaque, as is known in the art.

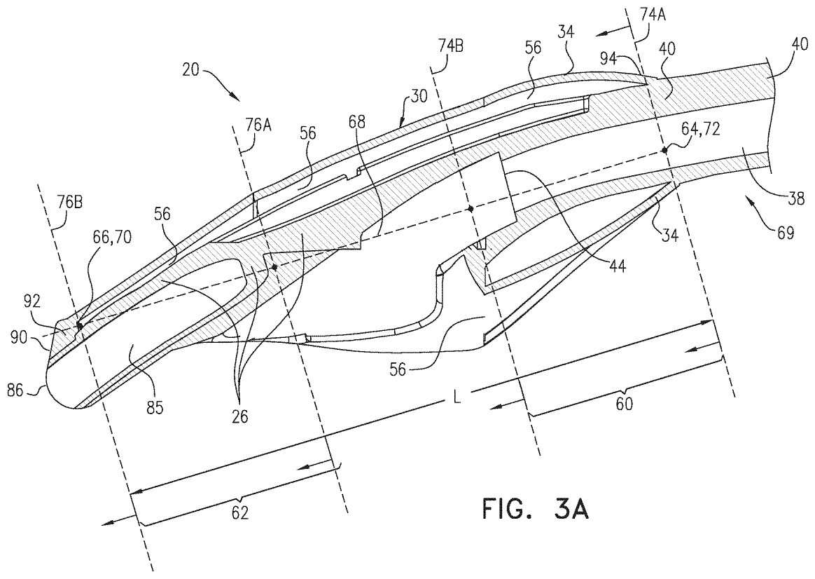

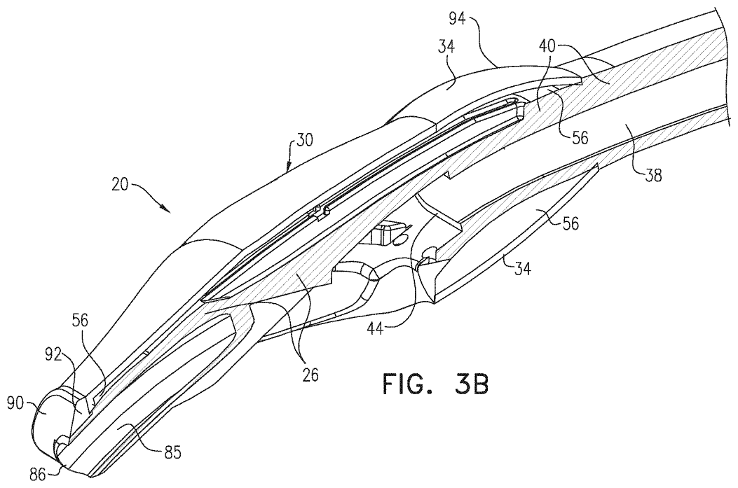

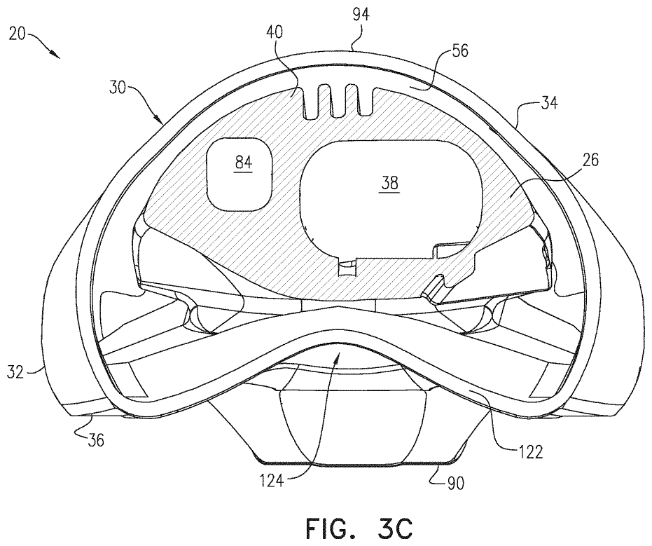

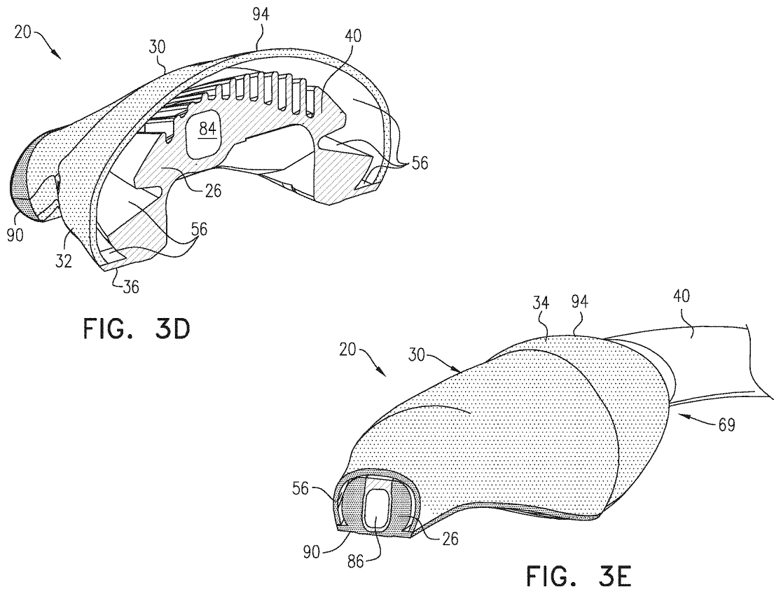

Reference is also made to FIGS. 3A-E, which are schematic cross-sectional views of laryngeal mask airway device 20, in accordance with an application of the present invention.

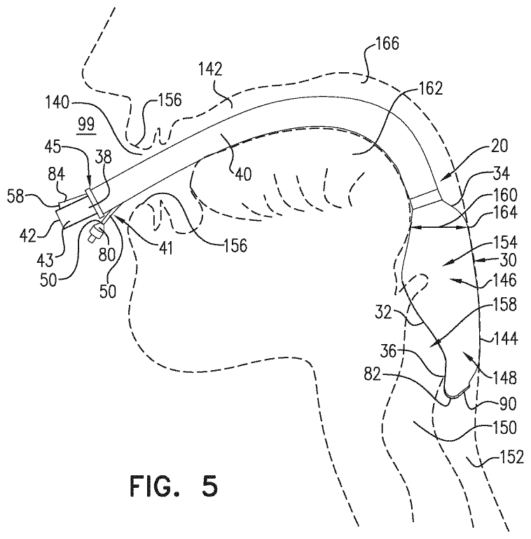

As mentioned above, laryngeal mask airway device 20 comprises inflatable balloon 30. Inflatable balloon 30 is shaped so as to define at least an inflatable annular cuff 32 and a proximal inflatable pharyngeal portion 34. Inflatable balloon 30 is insertable through an anterior opening 140 of an oral cavity 142 of a patient, as described hereinbelow with reference to FIG. 5. Inflatable annular cuff 32 shaped so as to define a cuff anterior side 36 that is configured to stabilize inflatable annular cuff 32 with respect to a laryngeal inlet 158 of the patient when inflatable balloon 30 is disposed at an insertion location 154 within a throat 144 of the patient, as described hereinbelow with reference to FIG. 5. Typically, inflatable annular cuff 32 and proximal inflatable pharyngeal portion 34 are contiguous portions of inflatable balloon 30, i.e., a continuous wall of a single piece of material defines both inflatable annular cuff 32 and proximal inflatable pharyngeal portion 34, without any internal separation between inflatable annular cuff 32 and proximal inflatable pharyngeal portion 34.

For some applications, inflatable balloon 30 comprises a highly elastic material, such as silicone, latex, or TPE. For these applications, inflatable balloon 30 may optionally have a Shore hardness of between oo-20 and A-30, such as between oo-20 and A-20. For other applications, inflatable balloon 30 comprises a material having minimal elasticity as commonly used in medical device cuff technology, e.g., PVC or polyurethane. For these applications, inflatable balloon 30 may optionally have a Shore hardness of between OO-30 and A-15.

For some applications, cuff anterior side 36 of inflatable annular cuff 32 has an average wall thickness that equals at least 200% (e.g., at least 300%, such as at least 500%) of an average wall thickness of all portions of inflatable balloon 30 other than cuff anterior side 36 of inflatable annular cuff 32. Alternatively or additionally, for some applications:
cuff anterior side 36 of inflatable annular cuff 32 has an average wall thickness of between 1 and 5 mm,
all portions of inflatable balloon 30 other than cuff anterior side 36 of inflatable annular cuff 32 have an average wall thickness of between 0.3 and 1.2 mm, and
proximal inflatable pharyngeal portion 34 has an average wall thickness of between 0.3 and 1.2 mm.

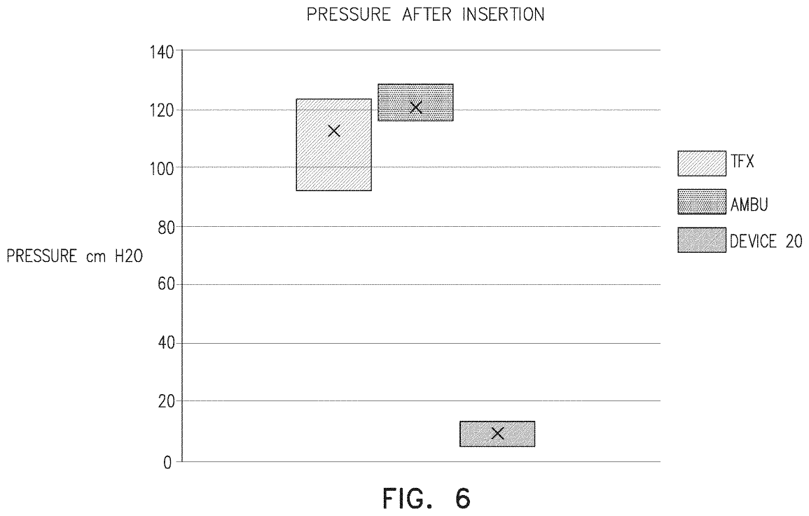

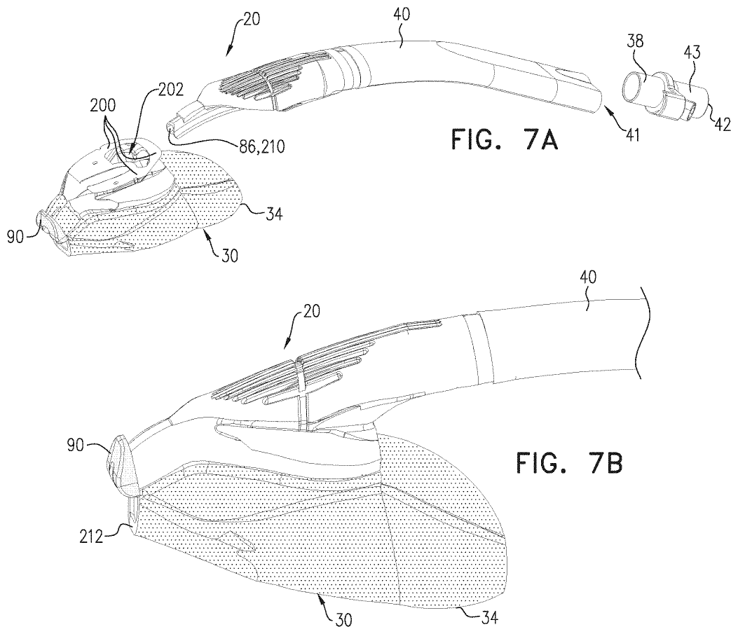

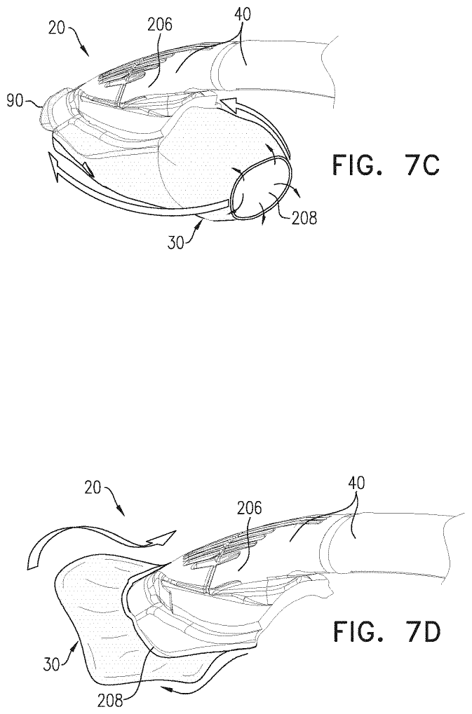

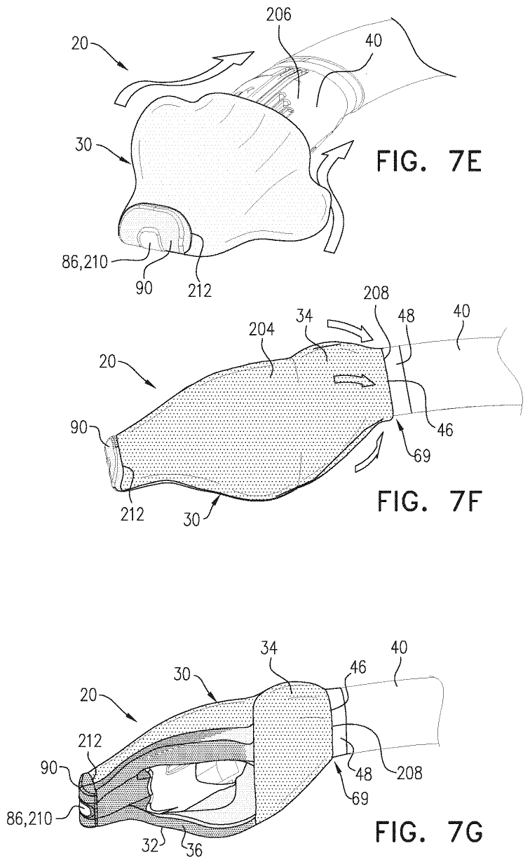

Laryngeal mask airway device 20 further comprises a shaft 40, which is installed through anterior opening 140 of oral cavity 142. Shaft 40 has (a) a proximal end 41 that is configured to be disposed outside the patient's oral cavity 142 when inflatable balloon 30 is disposed at insertion location 154, and (b) a distal end portion that is fixed to inflatable balloon 30. For some applications in which laryngeal mask airway device 20 comprises drainage tube 84, such as described hereinbelow, a distal end 210 (labeled in FIGS. 7A and 7G) of shaft 40 is shaped so as to define distal gastric opening 86. For other applications, distal end 210 may define a portion of distal tip portion 90, described hereinbelow, in order to provide support to the distal tip portion, or distal end 210 may be disposed more proximally within inflatable balloon 30 (e.g., more proximally within inflatable annular cuff 32). Typically, a proximal portion of shaft 40 is disposed outside the patient's oral cavity 142 when inflatable balloon 30 is disposed at insertion location 154, so as to serve as a handle 45 for inserting laryngeal mask airway device 20 into the patient's oral cavity 142 and positioning laryngeal mask airway device 20 properly at insertion location 154.

Laryngeal mask airway device 20 further comprises an airway tube 38, which passes through at least a longitudinal portion of shaft 40, such as through an entirety of a length of shaft 40, as shown in the drawings. For some applications, shaft 40 is shaped, e.g., molded, so as to define at least a portion of airway tube 38 within the shaft.

Airway tube 38 has (a) a proximal end 42 that is configured to be disposed outside the patient's oral cavity 142 when inflatable balloon 30 is disposed at insertion location 154, and (b) a distal end 44 that is open through inflatable annular cuff 32. Proximal end 42 is typically defined by an airway connector port 43, which is configured for connection to air or other ventilating apparatus for the patient's lungs.

For some applications, elongate shaft 40 comprises a PVC, silicone, or TPE. For these applications, elongate shaft 40 may optionally have a Shore A hardness of at least 30, typically between A40 and A80.

Proximal inflatable pharyngeal portion 34 of inflatable balloon 30 forms an air-tight seal 46 with an external surface 48 of shaft 40 (such as by gluing or welding). For some applications, inflatable balloon 30 and shaft 40 are configured such that proximal inflatable pharyngeal portion 34 of inflatable balloon 30 forms air-tight seal 46 entirely around a longitudinal portion 69 of shaft 40. For example, longitudinal portion 69 may have a length, measured along airway tube 38, of between 0.1 and 10 mm.

For some applications, proximal inflatable pharyngeal portion 34 extends in both lateral directions away from a straight line segment 68 (labeled in FIGS. 1C, 1G, and 3A). Straight line segment 68 connects (a) a distal centroid 70 of a distal end 66 of an inflatable chamber 56 defined by inflatable balloon 30 and (b) a proximal centroid 72 of proximal end 64 of inflatable chamber 56. Because of this dual lateral extension, proximal inflatable pharyngeal portion 34 provides support on both sides of the pharynx to prevent side-to-side tipping and/or tilting of laryngeal mask airway device 20. Typically, though not necessarily, the lateral portions of proximal inflatable pharyngeal portion 34 do not provide sealing with the throat in configurations of applications of the present invention.

For some applications, proximal inflatable pharyngeal portion 34 is reflectionally symmetric about a plane of symmetry that extends in an anterior/posterior direction, straight line segment 68 (labeled in FIGS. 1C, 1G, and 3A) falling in the plane of symmetry. (When inflatable balloon 30 is disposed at insertion location 154, the plane of symmetry corresponds to the sagittal plane of the patient.) Because of this reflectional symmetry, proximal inflatable pharyngeal portion 34 provides support on both sides of the pharynx to prevent side-to-side tipping and/or tilting of laryngeal mask airway device 20.

Laryngeal mask airway device 20 further comprises an externally-accessible inflation tube 50, for supplying air to and extracting air from the inflatable balloon 30. Inflation tube 50 has (a) a proximal end 52 that is configured to be disposed outside the patient's oral cavity 142 when inflatable balloon 30 is disposed at insertion location 154, and (b) a distal end that is coupled in fluid communication with inflatable chamber 56, for supplying air to the inflatable chamber. (For clarity of illustration, inflation tube 50 is shown only in FIGS. 1A, 1G, 1H, 2D, and 5, although it is provided in all configuration.)

For some applications, inflation tube 50 comprises an inflation tube proximal port connector that comprises a male conical fitting with a taper. For some applications, the taper is at least a 5% taper. For some applications, the taper is a 6% taper, and the male conical fitting with the 6% taper complies with International Standard ISO 594-1:1986, which is the standard for connections to conventional inflation lumen proximal ports of laryngeal mask airway masks.

For some applications, inflation tube 50 comprises an inflation valve 80 that is configured to be disposed outside the patient's oral cavity 142 when inflatable balloon 30 is disposed at insertion location 154, for holding a given inflation of inflatable balloon 30. Optionally, inflation valve 80 comprises a check-valve.

Typically, a distal portion of shaft 40 extends into inflatable chamber 56, such that a portion of external surface 48 of shaft 40 is in fluid communication with inflatable chamber 56 (external surface 48 is external with respect to shaft 40, but not necessarily with respect to other elements of laryngeal mask airway device 20, such as inflatable balloon 30). The distal portion of shaft 40 typically passes through at least a portion, such as all, of proximal inflatable pharyngeal portion 34 of inflatable balloon 30, such that, when inflatable chamber 56 is filled with the air at the pressure of 10 cm H2O and unconstrained, a portion of inflatable chamber 56 defined by proximal inflatable pharyngeal portion 34 surrounds 360 degrees of the distal portion of shaft 40. Optionally, a distal portion of shaft 40 may be considered to define a backplate.

For some applications, as labeled in FIGS. 1C, 1G, and 3A, inflatable balloon 30 is configured such that when inflatable chamber 56 is filled with air at a pressure of 10 cm H2O and unconstrained, a volume of the air in a proximal one-third 60 of inflatable chamber 56 equals at least 200% of a volume of the air in a distal one-third 62 of inflatable chamber 56, such as at least 300%, e.g., at least 350%, such as at least 400% of the volume of the air in distal one-third 62 of inflatable chamber 56. Proximal one-third 60 of inflatable chamber 56 extends distally from a proximal end 64 of inflatable chamber 56 along one third of a length L of inflatable chamber 56. Distal one-third 62 of inflatable chamber 56 extends proximally from distal end 66 of inflatable chamber 56 along one third of the length L of inflatable chamber 56. The length L is measured along straight line segment 68 connecting (a) a distal centroid 70 of distal end 66 of inflatable chamber 56 and (b) a proximal centroid 72 of proximal end 64 of inflatable chamber 56. (Proximal and distal planar edges 74A and 74B of proximal one-third 60, and proximal and distal planar edges 76A and 76B of distal one-third 62 of inflatable chamber 56, are perpendicular to straight line segment 68.) Typically, proximal inflatable pharyngeal portion 34 of inflatable balloon 30 provides at least a portion of, such as all of, proximal one-third 60 of inflatable chamber 56.

Alternatively or additionally, for some applications, inflatable balloon 30 is configured such that when inflatable chamber 56 is filled with air at a pressure of 20 cm H2O and unconstrained, the volume of the air in proximal one-third 60 of inflatable chamber 56 equals at least 200% of a volume of the air in a distal one-third 62 of inflatable chamber 56, such as at least 300%, e.g., at least 350%, such as at least 400% of the volume of the air in distal one-third 62 of inflatable chamber 56.

Further alternatively or additionally, for some applications, inflatable balloon 30 is configured such that when inflatable chamber 56 is filled with air at a pressure of 25 cm H2O and unconstrained, the volume of the air in proximal one-third 60 of inflatable chamber 56 equals at least 200% of a volume of the air in a distal one-third 62 of inflatable chamber 56, such as at least 300%, e.g., at least 350%, such as at least 400% of the volume of the air in distal one-third 62 of inflatable chamber 56.

For some applications, inflatable balloon 30 is configured such that when inflatable chamber 56 is filled with the air at the pressure of 10 cm H2O and unconstrained:
the volume of the air in proximal one-third 60 of inflatable chamber 56 is at least 5 cc, such as at least 10 cc, and/or no more than 50 cc, and/or
the volume of the air in distal one-third 62 of inflatable chamber 56 is less than 10 cc, such as less than 5, and/or at least 2 cc.

Alternatively or additionally, for some applications, inflatable balloon 30 is configured to have the volumes mentioned immediately above when inflatable chamber 56 is filled with the air at a pressure of 20 cm H2O and unconstrained.

Further alternatively or additionally, for some applications, inflatable balloon 30 is configured to have the volumes mentioned immediately above when inflatable chamber 56 is filled with the air at a pressure of 25 cm H2O and unconstrained.

As used in the present application, including in the claims and the Inventive Concepts, “unconstrained” means not constrained by the patient's anatomy, a delivery tool, or anything else.

For some applications, inflatable annular cuff 32 is shaped so as to define a distal tip portion 90 that includes a distal end 82 of inflatable annular cuff 32. Distal tip portion 90 is configured to form a seal against an upper esophageal sphincter, typically at the level of the cricoid cartilage ring, or the first tracheal cartilage ring immediately below the cricoid cartilage ring when inflatable balloon 30 is disposed at insertion location 154. Typically, a distal wall 92 (labeled in FIGS. 2A, 3A, and 3B) of the distal tip portion has a thickness of at least 2 mm, such as at least 4 mm, e.g., at least 6 mm, e.g., 8 mm. For some applications, distal tip portion 90 and proximal inflatable pharyngeal portion 34 of inflatable balloon 30 together seal esophagus 152, such as for applications in which the anterior side of laryngeal mask airway device 20 is not configured to form a seal around laryngeal inlet 158 of the patient when inflatable annular cuff 32 is disposed at insertion location 154, such as described hereinbelow.

In some applications of the present invention, such as shown in the figures, laryngeal mask airway device 20 is of the gastro-laryngeal mask (GLM) type, in which a drainage tube 84 (a) extends from a gastric channel 85 (labeled in FIGS. 3A and 3B) having a distal gastric opening 86 through distal tip portion 90, and (b) has a proximal drainage port 58 at location outside the patient's oral cavity when inflatable balloon 30 is disposed at insertion location 154. Drainage tube 84 enables extraction and external removal of gastric-discharge products from esophagus 152. Such GLM devices are commonly used in hospital settings in which evacuation suction sources are available, while simpler configurations of laryngeal mask airway devices without evacuation tubes are more commonly used in emergency intubations settings. Although the figures show drainage tube 84, drainage tube 84 is not an essential element of laryngeal mask airway device 20, and is not provided in some embodiments of the invention. Therefore, unless specifically stated to the contrary, all features of laryngeal mask airway device 20 described herein apply to laryngeal mask airway designs both with and without drainage tube 84.

Reference is again made to FIGS. 3A-B. For some applications, laryngeal mask airway device 20 comprises an inner support 26, which is mostly (typically entirely) covered by inflatable balloon 30. Inner support 26 provides additional structure to laryngeal mask airway device 20. Typically, inner support 26 comprises a material that has a greater hardness than the material of inflatable balloon 30. For example, inner support 26 may optionally have a Shore hardness of at least A-20, such as at least A-30, e.g., at least A-50. For example, inner support 26 may comprise silicone, latex, or TPE. Typically, inner support 26 extends distally from elongate shaft 40. Optionally, inner support 26 and elongate shaft 40 are fabricated from a single piece of material.

For some applications in which laryngeal mask airway device 20 is of the GLM type, as described above, inner support 26 is shaped so as to define gastric channel 85 therethrough.

For some applications, laryngeal mask airway device 20 is configured such that when inflatable chamber 56 is filled with the air at the pressure of 10 cm H2O and unconstrained, a portion 94 of proximal inflatable pharyngeal portion 34 of inflatable balloon 30 bulges posteriorly with respect to external surface 48 of shaft 40.

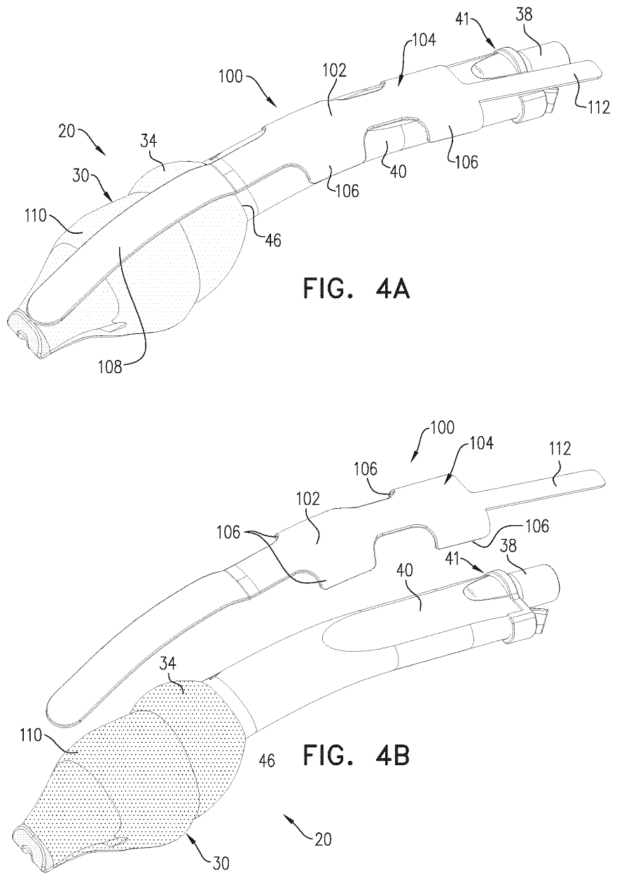

Reference is now made to FIGS. 4A-B, which are schematic illustrations of an insertion posterior protector 100 coupled to and decoupled from laryngeal mask airway device 20, respectively, in accordance with an application of the present invention. Insertion posterior protector 100 comprises an elongate sheet of material 102 that is shaped so as to define:
a proximal coupling portion 104, which is shaped so as to define one or more couplers 106 that are configured to removably couple the proximal coupling portion to shaft 40 proximally to inflatable balloon 30, and
a distal protection portion 108, which is shaped so as to partially cover a posterior external surface 110 of inflatable balloon 30 when proximal coupling portion 104 is removably coupled to shaft 40, so as to protect inflatable balloon 30 from puncture (such as by the patient's teeth) during insertion thereof into the patient's oral cavity 142.

For some applications, the one or more couplers 106 are shaped so as to form snap-on couplings to shaft 40, such as illustrated. For other applications, the one or more couplers 106 are shaped as to otherwise mechanically or chemically removably couple the proximal coupling portion to shaft 40 proximally to inflatable balloon 30.

For some applications, elongate sheet of material 102 of insertion posterior protector 100 is further shaped so as to define a proximal handle 112, which is shaped so as to be disposed outside the patient's oral cavity 142 when inflatable balloon 30 is disposed at insertion location 154. Proximal handle 112 may be used to decouple insertion posterior protector 100 from shaft 40 and remove insertion posterior protector 100 from the patient's oral cavity 142 after inflatable balloon 30 has been disposed at insertion location 154.

For some applications, elongate sheet of material 102 has an average thickness of between 0.5 and 2 mm. For some applications, elongate sheet of material 102 comprises a polymer (e.g., PVC, ABS, or polyurethane) or a metal.

Reference is now made to FIGS. 1H and 2D, which are schematic cross-sectional proximal views of laryngeal mask airway device 20. For some applications, cuff anterior side 36, along a proximal side 122 of inflatable annular cuff 32, is shaped so as to define a posteriorly-recessed portion 124, i.e., a portion that is recessed in a posterior direction. Optionally, posteriorly-recessed portion 124 of cuff anterior side 36 is curved. Posteriorly-recessed portion 124 prevents inflatable annular cuff 32 from applying excessive pressure to a posterior portion of the patient's tongue, which might cause tissue damage.

Reference is now made to International Application PCT/IL2019/050405, filed Apr. 11, 2019, which published as PCT Publication WO 2019/198081, and to FIG. 12C of U.S. application Ser. No. 16/423,974, filed May 28, 2019, which published as US Patent Application Publication 2019/0282774, and which is a continuation of the '405 PCT application. Both of these applications are assigned to the assignee of the present application and are incorporated herein by reference.

As shown in the figures of these two patent applications, in particular in FIG. 12C, a laryngeal mask airway device 900 is provided that comprises an inflatable balloon 912, which is shaped so as to define at least an inflatable annular cuff 910. Inflatable balloon 912 is insertable through an anterior opening of an oral cavity of a patient. Inflatable annular cuff 910 is shaped so as to define a cuff anterior side that is configured to surround a laryngeal inlet of the patient when inflatable annular cuff 910 is disposed at an insertion location within a throat of the patient. The cuff anterior side, along a proximal side of inflatable annular cuff 910, is shaped so as to define a posteriorly-recessed portion.

For some applications, the posteriorly-recessed portion is curved, such as shown in FIG. 12C of these two patent applications.

Laryngeal mask airway device 900 of these two patent applications further comprises:
an airway tube 125, which extends proximally from inflatable balloon 912, and which has (a) a proximal end 120 that is configured to be disposed outside the patient's oral cavity when inflatable annular cuff 910 is disposed at insertion location 154, and (b) a distal end that is open through inflatable annular cuff 910; and
an inflation tube 129 having (a) a proximal end that is configured to be disposed outside the patient's oral cavity when inflatable annular cuff 910 is disposed at an insertion location, and (b) a distal end that is coupled in fluid communication with an inflatable chamber of inflatable balloon 912, for supplying air to the inflatable chamber.

For some applications, the anterior side of laryngeal mask airway device 900 of these two patent applications is configured to form a seal around laryngeal inlet 158 of the patient when inflatable annular cuff 910 is disposed at the insertion location.

Reference is now made to FIG. 5 of the present application, which is a schematic illustration of laryngeal mask airway device 20 inserted through anterior opening 140 of oral cavity 142 of a patient, in accordance with an application of the present invention. In typical use, laryngeal mask airway device 20 should be inserted into throat 144 while inflatable balloon 30 is deflated; however, in practice, laryngeal mask airway devices are often inserted while inflated at ambient pressure, such as described hereinbelow with reference to FIG. 6. Laryngeal mask airway device 20 is lodged in a pharynx 146 at the base of a hypopharynx 148 where throat 144 divides into a trachea 150 and an esophagus 152, such that inflatable balloon 30 is disposed at insertion location 154 within throat 144. (Anterior opening 140 is the opening of the mouth between lips 156, between outside oral cavity 142 and inside oral cavity 142.) Laryngeal mask airway device 20 is typically used for ventilating lungs of the patient.

For some applications, cuff anterior side 36 is configured to form a seal around laryngeal inlet 158 of the patient when inflatable balloon 30 is disposed at insertion location 154. Alternatively, the anterior side of laryngeal mask airway device 20 is not configured to form a seal around laryngeal inlet 158 of the patient when inflatable annular cuff 32 is disposed at insertion location 154 thereby causing less irritation of soft tissue and causing inflatable annular cuff 32 to be less sensitive to inter-personal variations of anatomy.

For some applications, laryngeal mask airway device 20 is configured such that when inflatable balloon 30 is disposed at insertion location 154 and proximal inflatable pharyngeal portion 34 of inflatable balloon 30 is inflated at the pressure of 10 cm H2O, inflatable annular cuff 32 spans both (a) a vertical space 160 between a tongue 162 and a pharyngeal back wall 164 of throat 144 and (b) a lateral space between left and right tonsils, thereby forming a proximal seal between oral cavity 142 and a pharyngeal space 166 above the glottis, distal to proximal inflatable pharyngeal portion 34 of inflatable balloon 30.

Reference is still made to FIG. 5, and is additionally made to FIG. 6, which is a graph showing pressures measured within prior art laryngeal mask airway cuffs and inflatable balloon 30 of laryngeal mask airway device 20, as measured during an experiment conducted by the inventor of the present invention.

The experiment measured pressures within the cuff/balloon of the following laryngeal mask airway devices after insertion of the cuff/balloon into an adult doll ventilation-simulation model:
an LMA Supreme™ size #4 laryngeal mask device cuff (Teleflex Inc., Wayne, Pa., USA), having a Shore hardness of A50 (labeled “TFX” in FIG. 6);
an Ambu® AuraOnce™ size #4 laryngeal mask device cuff (Ambu A/S, Ballerup, Denmark), comprising PVC and having a Shore hardness of greater than A40 (labeled “AMBU” in FIG. 6); and
a laryngeal mask airway device similar to laryngeal mask airway device 20 described herein, comprising silicone and having a Shore hardness of OO-38 (labeled “DEVICE 20” in FIG. 6).

All the cuffs/balloons were inflated to ambient room pressure prior to insertion. They were then inserted into the doll model, while their respective inflation valves were closed. Pressure was measured after insertion. The graph shows the full range of outcomes for each device. As can be seen, the pressures measured in the Teleflex and AMBU cuffs were significantly above 60 cm H2O. By contrast, the pressure measured in the laryngeal mask airway device similar to laryngeal mask airway device 20 described herein was significantly below 30 cm H2O.

These experimental results show that the design of laryngeal mask airway device 20, unlike those of the prior art cuffs tested, prevents excess pressure from developing in the cuff/balloon, even when the cuff/balloon is inserted with the inflation valve closed, and the cuff/balloon has not been deflated. The inventor believes that in practice many users insert laryngeal mask airway devices without deflating and without opening the inflation valve, despite explicit user instructions to deflate before insertion, and then reinflate after insertion to a proper pressure below 60 cm H2O. Many users also do not measure the pressure after insertion, also despite user instructions requiring this safety procedure. As is known in the art, pressures over 40 cm H2O may cause tissue damage if the cuff/balloon remains inserted for more than 60 minutes.

Particular aspects of the design of laryngeal mask airway device 20 that may contribute to prevention of this excess pressure include (a) the relatively high ratio of the volume of the air in proximal one-third 60 of inflatable chamber 56 to the volume of the air in distal one-third 62 of inflatable chamber 56, described hereinabove with reference to FIGS. 1C, 1G, and 3A, which causes the proximal one-third 60 to function as an excess air pressure absorber, (b) the consequent relatively low volume in distal one-third 62 of inflatable chamber 56, and (c) the softness of the material of inflatable balloon 30.

For some applications, laryngeal mask airway device 20 is configured such that when inflatable balloon 30, while inflation valve 80 is closed and the pressure within inflatable balloon 30 equals ambient pressure of ambient air 99 (labeled in FIG. 5), is inserted from outside oral cavity 142 of the patient to insertion location 154 within throat 144 without opening inflation valve 80, the pressure within inflatable balloon 30 increases from the ambient pressure to an inserted-location pressure, the inserted-location pressure less than 60 cm H2O, such as less than 45 cm H2O, e.g., less than 30 cm H2O. The ability of laryngeal mask airway device 20 to be thus configured is supported by the experimental results described hereinabove with reference to FIG. 6.

In an application of the present invention, a method is provided that comprises:
inserting inflatable balloon 30 of laryngeal mask airway device 20 through anterior opening 140 of oral cavity 142 and disposing inflatable balloon 30 at insertion location 154 within throat 144, such that (a) proximal end 52 of inflation tube 50 is disposed outside oral cavity 142, (b) inflation valve 80 of inflation tube 50 and proximal end 42 of airway tube 38 are disposed outside oral cavity 142, and (c) an anterior side of laryngeal mask airway device 20 stabilizes laryngeal mask airway device 20 with respect to laryngeal inlet 158 of the patient; and
ventilating lungs of the patient using laryngeal mask airway device 20.

For some applications, inserting inflatable balloon 30 comprises inserting inflatable balloon 30, while inflation valve 80 is closed, from outside oral cavity 142 to insertion location 154 within throat 144 without opening inflation valve 80. For some applications, laryngeal mask airway device 20 is configured such that when inflatable balloon 30, while inflation valve 80 is closed and a pressure within inflatable balloon 30 equals ambient pressure, is inserted from outside oral cavity 142 to insertion location 154 within throat 144 without opening inflation valve 80, the pressure within inflatable balloon 30 increases from the ambient pressure to an inserted-location pressure, the inserted-location pressure less than 60 cm H2O, such as less than 45 cm H2O, e.g., less than 30 cm H2O.

For some applications, inserting inflatable balloon 30 while inflation valve 80 is closed comprises inserting inflatable balloon 30 while inflation valve 80 is closed and the pressure within inflatable balloon 30 equals ambient pressure.

For some applications, inserting inflatable balloon 30 while inflation valve 80 is closed comprises inserting inflatable balloon 30 while inflation valve 80 is closed without deflating inflatable balloon 30 before inserting inflatable balloon 30.

For some applications, the method does not comprise measuring the pressure within inflatable balloon 30. For other applications, the pressure is measured.

For some applications, the method does not comprise adding air to or removing air from inflatable balloon 30 after inserting inflatable balloon 30 to insertion location 154. For other applications, air is added to or removed from inflatable balloon 30 after inserting inflatable balloon 30 to insertion location 154, such as for applications in which the pressure is measured, as described above, and found to be too low or too high, respectively.

For some applications, the method does not comprise adjusting the pressure within inflatable balloon 30 after inserting inflatable balloon 30 to insertion location 154. For other applications, the pressure is adjusted within inflatable balloon 30 after inserting inflatable balloon 30 to insertion location 154, such as for applications in which the pressure is measured, as described above, and found to be too low or too high.

Reference is now made to FIGS. 7A-G, which are schematic illustrations of a method of manufacturing a laryngeal mask airway device, in accordance with an application of the present invention. Although the method is illustrated for manufacturing laryngeal mask airway device 20, the method may alternatively be used for manufacturing other laryngeal mask airway devices that are similar in some respects to laryngeal mask airway device 20, mutatis mutandis.

FIG. 7A shows components of laryngeal mask airway device 20. Inflatable balloon 30 may be formed (e.g., molded) from a single piece of material, as shown, or from two or more pieces of material that are fixed together, such as by gluing and/or welding. Inflatable balloon 30 is shown inverted in FIG. 7A. Inflatable balloon 30 may be formed (e.g., molded) in this inverted state, or may alternatively be formed (e.g., molded) in its final, non-inverted state and thereafter inverted to arrive at the inverted state shown in FIG. 7A (as well FIGS. 7B and 7C). (Of course, inflatable balloon 30 cannot be inflated when in this inverted manufacturing state.)

As shown in FIG. 7B, inflatable balloon 30 is attached (such as by gluing and/or welding) to shaft 40 while inflatable balloon 30 is inverted, by sealing at least a portion of a perimeter 200 of a lateral opening 202 of inverted inflatable balloon 30 to shaft 40, such that inverted inflatable balloon 30 extends anteriorly from shaft 40.

As shown in FIGS. 7C-F, thereafter, inverted inflatable balloon 30 is inverted such that a posterior portion 204 of inflatable balloon 30 covers at least a portion of a posterior region 206 of shaft 40.

Typically, thereafter, inflatable balloon 30 (such as proximal inflatable pharyngeal portion 34 thereof, if provided) is sealed with air-tight seal 46 with external surface 48 of shaft 40 (such as by gluing or welding). For some applications, inflatable balloon 30 forms air-tight seal 46 entirely around longitudinal portion 69 of shaft 40.

For some applications in which inflatable annular cuff 32 is shaped so as to define distal tip portion 90, a distal end opening 212 of inflatable balloon 30 is sealed to distal tip portion 90 after inflatable balloon 30 has been inverted.

For some applications, inverted inflatable balloon 30 is shaped so as to define a proximal end opening 208, which, after the inverted inflatable balloon is attached to shaft 40 and before inflatable balloon 30 is inverted, such as shown in FIGS. 7A-C, is not in direct contact with any portions of laryngeal mask airway device 20 other than inverted inflatable balloon 30. For these applications, inverted inflatable balloon 30 is inverted by passing, in a distal-to-proximal direction, proximal end opening 208 of inflatable balloon 30 over and around distal end 210 (labeled in FIGS. 7A and 7G) of shaft 40, until proximal end opening 208 of inflatable balloon 30 surrounds shaft 40. (For some applications in which laryngeal mask airway device 20 comprises drainage tube 84, distal end of shaft 40 is shaped so as to define distal gastric opening 86.) After inverted inflatable balloon 30 is inverted, proximal end opening 208 of inflatable balloon 30 is sealed with air-tight seal 46 with external surface 48 of shaft 40.

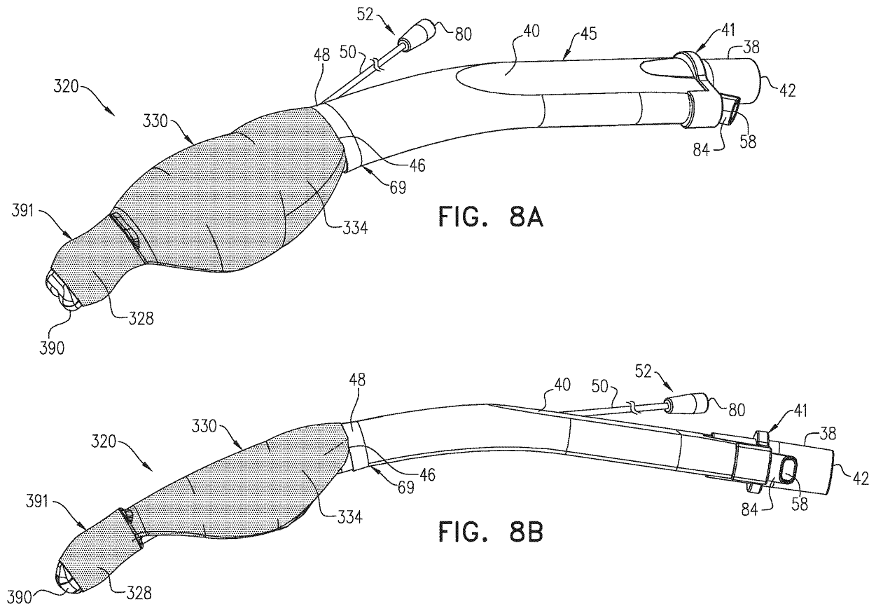

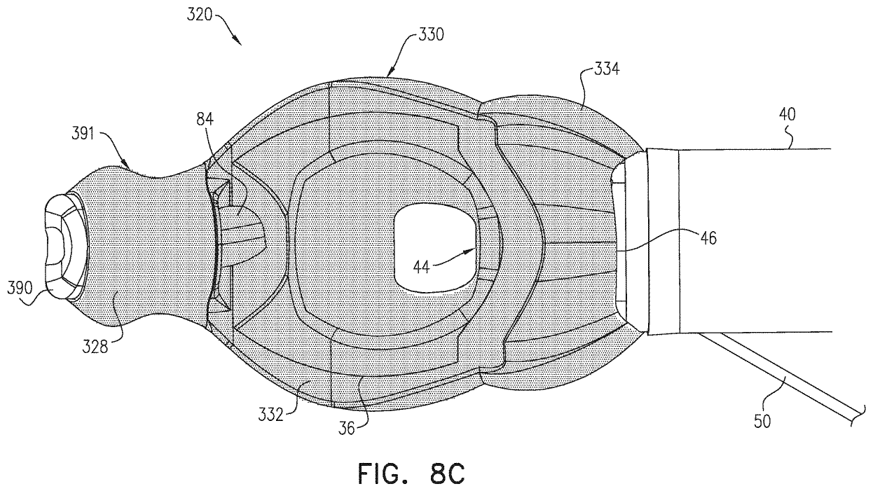

Reference is made to FIGS. 8A-C, which are schematic illustrations of several views of a laryngeal mask airway device 320, in accordance with an application of the present invention.

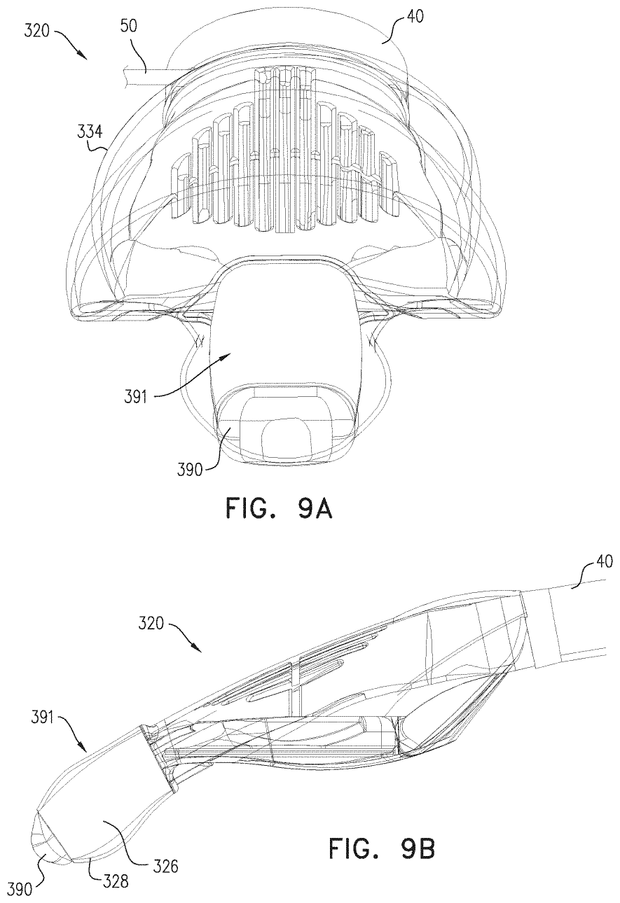

Reference is also made to FIGS. 9A-B, which are schematic illustrations of several views of laryngeal mask airway device 320, in accordance with an application of the present invention. In FIGS. 9A-B, a portion of an inflatable balloon 330 of laryngeal mask airway device 320 is shown as translucent to show some elements the view of which would otherwise be blocked by the portion of inflatable balloon 330. In practice, inflatable balloon 330 may be translucent, transparent, or opaque, as is known in the art.

Reference is also made to FIGS. 10A-B, which are schematic cross-sectional views of laryngeal mask airway device 320, in accordance with an application of the present invention.

Other than as described hereinbelow, laryngeal mask airway device 320 is generally similar to laryngeal mask airway device 20, described hereinabove with reference to FIGS. 1A-7G, and may implement any of the features thereof, mutatis mutandis, including, but not limited to, the relative volumes of air in the proximal and distal one-thirds of inflatable chamber 56. Like reference numerals refer to like parts.

Inflatable balloon 330 is shaped so as to define at least an inflatable annular cuff 332 (labeled in FIG. 8C) and a proximal inflatable pharyngeal portion 334. Inflatable balloon 330 is insertable through anterior opening 140 of oral cavity 142, as described hereinabove with reference to FIG. 5 regarding laryngeal mask airway device 20 regarding laryngeal mask airway device 20. Inflatable annular cuff 332 and proximal inflatable pharyngeal portion 334 may implement any of the features of inflatable annular cuff 32 and proximal inflatable pharyngeal portion 34, respectively, described hereinabove with reference to FIGS. 1A-7G, mutatis mutandis.

For some applications, laryngeal mask airway device 320 comprises a distal tip portion 391 located distal to inflatable annular cuff 332. Distal tip portion 391 is configured to form a seal against an upper esophageal sphincter when inflatable balloon 330 is disposed at insertion location 154. For some applications, distal tip portion 391 and proximal inflatable pharyngeal portion 334 of inflatable balloon 330 together seal esophagus 152, such as for applications in which the anterior side of laryngeal mask airway device 320 is not configured to form a seal around laryngeal inlet 158 of the patient when inflatable annular cuff 32 is disposed at insertion location 154.

For some applications, laryngeal mask airway device 320 comprises an inner support 326 (labeled in FIGS. 9B, 10A-B, and 13), which is mostly (but typically not entirely) covered by inflatable balloon 330. Inner support 326 provides additional structure to laryngeal mask airway device 320. Typically, inner support 326 comprises a material that has a greater hardness than the material of inflatable balloon 330. For example, inner support 326 may optionally have a Shore hardness of at least A-20, such as at least A-30, e.g., at least A-50. For example, inner support 326 may comprise silicone, latex, or TPE. Typically, inner support 326 extends distally from elongate shaft 40. Optionally, inner support 326 and elongate shaft 40 are fabricated from a single piece of material.

For some applications in which laryngeal mask airway device 320 is of the GLM type, as described above, inner support 26 is shaped so as to define gastric channel 85 therethrough.

For some applications, inflatable balloon 330 is shaped so as to define a distal inflatable tip portion 328, which is typically in fluid communication with the rest of inflatable chamber 56 defined by inflatable balloon 330 (labeled for inflatable balloon 30 in FIGS. 2A, 2B, and 2D). Typically, distal inflatable tip portion 328 entirely surrounds a longitudinal portion 378 of distal tip portion 391 (labeled in FIG. 10A). A distal end 396 of longitudinal portion 378 is at a distance D from a distal-most tip 398 of distal tip portion 391. Typically, the distance D is at least 1 mm, no more than 5 mm, and/or between 1 and 5 mm. Thus, distal tip portion 391 typically comprises a distal-most non-balloon-covered tip portion 390, defined by inner support 26. Typically, longitudinal portion 378 has a length L of at least 1 cm, no more than 5 cm, and/or between 1 and 5 cm, such as at least 2 cm, no more than 4 cm, and/or between 2 and 4 cm, e.g., 3 cm. Optionally, longitudinal portion 378 has a greatest width W, measured from side to side of inflatable balloon 330 (i.e., in the direction perpendicular to the cross-section shown in FIG. 10A), of no more than 20 mm, such as no more than 16 mm (e.g., of 12 mm) along the length L of longitudinal portion 378.

For some applications, a proximal end 388 of distal inflatable tip portion 328 is at least partially coupled (e.g., by gluing or welding) to inner support 326, so as to restrict air flow between distal inflatable tip portion 328 and inflatable annular cuff 332, without entirely blocking the air flow. This coupling may prevent distal inflatable tip portion 328 from being drawn too far proximally during insertion of distal tip portion 391 into esophagus 152. If not for this coupling, distal inflatable tip portion 328 might become so tightened that it does not inflate when inflatable balloon 330 is inflated after insertion of inflatable annular cuff 332 to insertion location 154. Thus this coupling results in distal inflatable tip portion 328 becoming inflated when inflatable annular cuff 332 is inflated after insertion.

Optionally, proximal end 388 of distal inflatable tip portion 328 is at least partially coupled to inner support 326 at a proximal end 389 of distal tip portion 391.

Optionally, an external surface of inner support 326 is shaped so as to define one or more indentations, such as elongate grooves, for providing fluid communication between inflatable annular cuff 332 and distal inflatable tip portion 328 at the location of coupling described above.

Reference is now made to FIG. 11, which is a schematic illustration of insertion posterior protector 100 coupled to laryngeal mask airway device 320, in accordance with an application of the present invention. Insertion posterior protector 100 and its use are described hereinabove with reference to FIGS. 4A-B for laryngeal mask airway device 20.

Reference is now made to FIG. 12, which is a graph showing pressures measured within prior art laryngeal mask airway cuffs and inflatable balloon 330 of laryngeal mask airway device 320, as measured during an experiment conducted on behalf of the inventor of the present invention.

The experiment measured pressures within the cuff/balloon of the following laryngeal mask airway devices:
an LMA Supreme™ size #4 laryngeal mask device cuff (Teleflex Inc., Wayne, Pa., USA);
an Ambu® AuraOnce™ size #4 laryngeal mask device cuff (Ambu A/S, Ballerup, Denmark), comprising PVC; and
a laryngeal mask airway device similar to laryngeal mask airway device 320 described herein, comprising silicone and having a Shore hardness of A10 (labeled “DEVICE 320” in FIG. 12).

All the cuffs/balloons were inflated to an inflation pressure of 30 cm H2O. Thereafter, 5 cc of ambient air was drawn into a syringe and introduced into each of the respective cuffs/balloons. The inflation pressure in the balloon of the laryngeal mask airway device similar to laryngeal mask airway device 320 increased from the initial 30 cm H2O to 33.1 cm H2O (an increase of about 3 cm H2O), while the inflation pressures in the cuffs/balloons of the Teleflex and Ambu® devices increased from the initial 30 cm H2O to 80.6 cm H2O and 111.2 cm H2O, respectively (increases of about 51 and 81 cm H2O, respectively).

These experimental results show that the design of laryngeal mask airway device 320, unlike those of the prior art cuffs tested, prevents excess pressure from developing in the cuff/balloon. This relatively low pressure increase upon moderate additional inflation reduces the risk of damage to soft tissue that might be caused by higher pressures within inflatable balloon 330. As is known in the art, pressures over 40 cm H2O may cause tissue damage if the cuff/balloon remains inserted for more than 60 minutes.

Particular aspects of the design of laryngeal mask airway device 320 that may contribute to prevention of this excess pressure include.

For some applications, laryngeal mask airway device 320 is configured such that when a volume of 5 cc of ambient air, the volume measured at standard atmospheric pressure, is introduced into externally-accessible inflation tube 50 while inflatable balloon 330 is unconstrained, disposed in ambient air that is at standard atmospheric pressure (i.e., air pressure at sea level), and at is an inflation pressure of 30 cm H2O:
the inflation pressure in inflatable balloon 330, measured one minute after introduction of the volume of 5 cc of the ambient air, is less than 40 cm H2O.

(Waiting the one minute allows the pressure in inflatable balloon 330 to stabilize.)

The ability of laryngeal mask airway device 320 to be thus configured is supported by the experimental results described hereinabove with reference to FIG. 12.

For some applications, laryngeal mask airway device 20, described hereinabove with reference to FIGS. 1A-7G, is configured such that when a volume of 5 cc of ambient air, the volume measured at standard atmospheric pressure, is introduced into externally-accessible inflation tube 50 while inflatable balloon 30 is unconstrained, disposed in ambient air that is at standard atmospheric pressure (i.e., air pressure at sea level), and at is an inflation pressure of 30 cm H2O:
the inflation pressure in inflatable balloon 30, measured one minute after introduction of the volume of 5 cc of the ambient air, is less than 40 cm H2O.

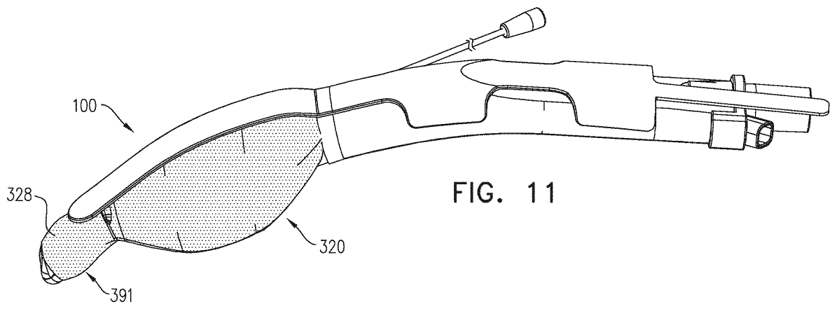

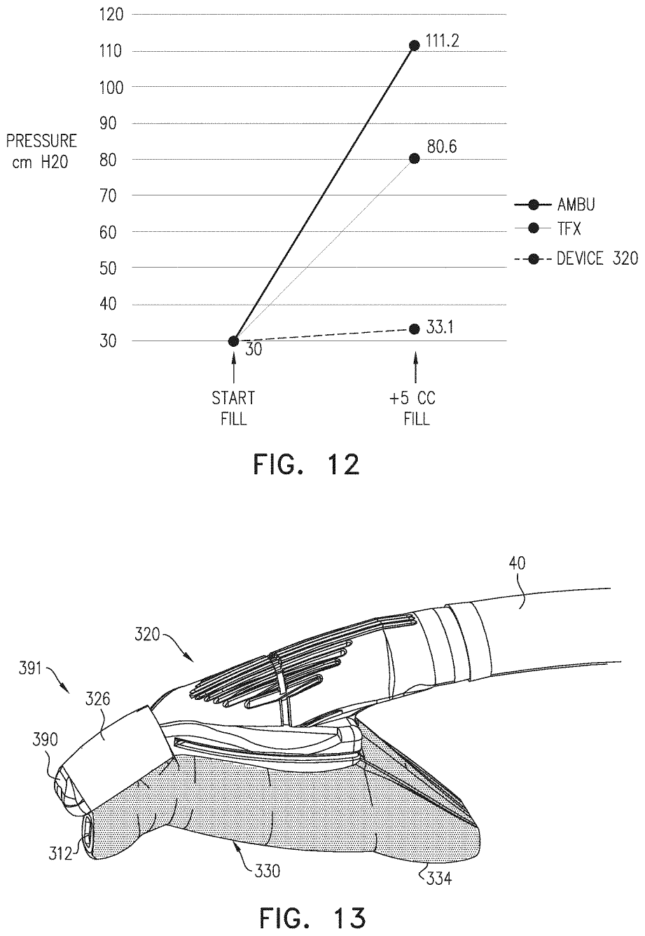

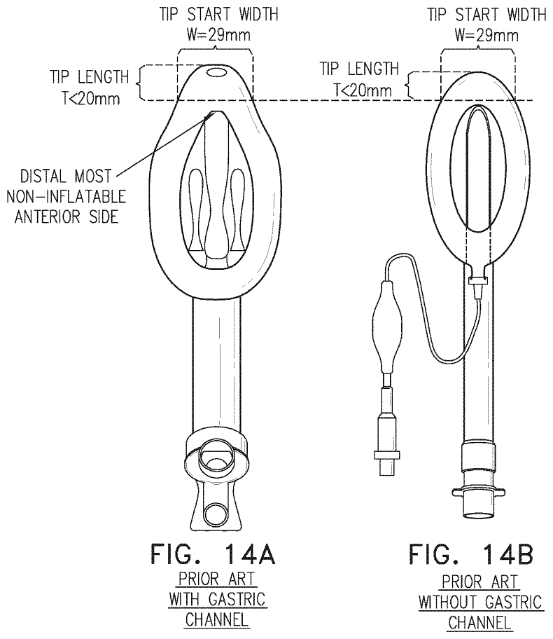

For some applications, laryngeal mask airway device 420, described hereinbelow with reference to FIGS. 15A-B, 17A-B, and 18, is configured such that when a volume of 5 cc of ambient air, the volume measured at standard atmospheric pressure, is introduced into externally-accessible inflation tube 50 while inflatable annular cuff 432 is unconstrained, disposed in ambient air that is at standard atmospheric pressure (i.e., air pressure at sea level), and at is an inflation pressure of 30 cm H2O:
the inflation pressure in inflatable annular cuff 432, measured one minute after introduction of the volume of 5 cc of the ambient air, is less than 40 cm H2O.

Reference is now made to FIG. 13, which is a schematic illustration of one stage of a method of manufacturing laryngeal mask airway device 320, in accordance with an application of the present invention. FIG. 13 illustrated, for laryngeal mask airway device 320, the same stage of manufacture described hereinabove with reference to FIG. 7B for laryngeal mask airway device 20. The manufacturing process may be similar to that described hereinabove with reference to FIGS. 7A-G, mutatis mutandis.

In the present application, including in the claims and the Inventive Concepts, all pressures are gauge pressures that are zero-referenced against ambient air 99 pressure.

Typically, but not necessarily, as is conventional in the laryngeal mask airway cuff art, inflatable balloons 30 and 330 (including inflatable annular cuffs 32 and 332) are single-layer balloons, i.e., have only a single wall, as can be seen in the cross-sectional figures. The wall thicknesses of inflatable balloons 30 and 330 and inflatable annular cuffs 32 and 332 described relate to the thickness of this single layer, i.e., single wall.

Reference is made to FIGS. 15A-B, which are schematic illustrations of a cuff anterior side 422 and a cuff posterior side 424, respectively, of a laryngeal mask airway device 420, in accordance with an application of the present invention.

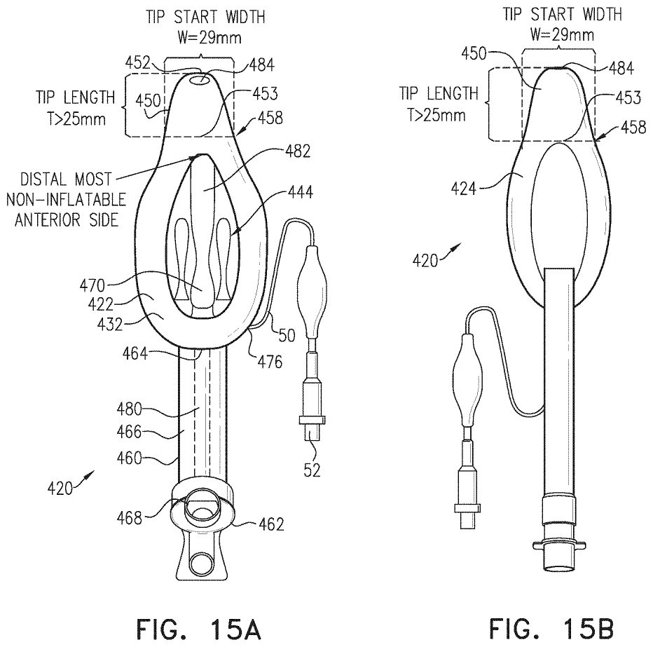

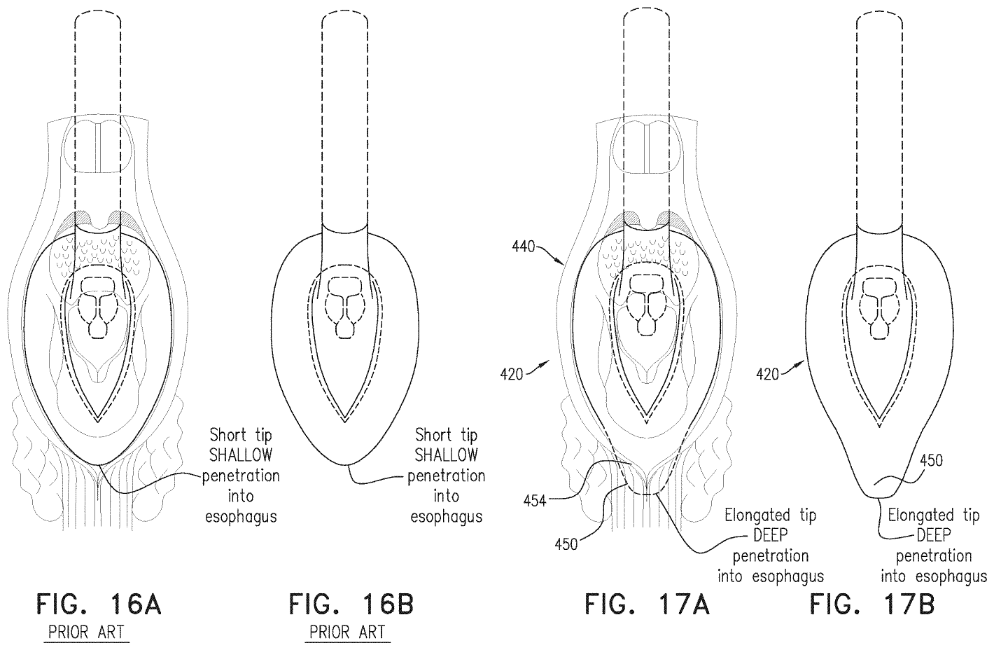

Reference is also made to FIGS. 17A-B, which are schematic illustrations of cuff anterior and posterior sides 422 and 424, respectively, of laryngeal mask airway device 420, in accordance with an application of the present invention. FIG. 17A shows an inflatable annular cuff 432 of laryngeal mask airway device 420 disposed at an insertion location 440 within throat 144 of a patient.

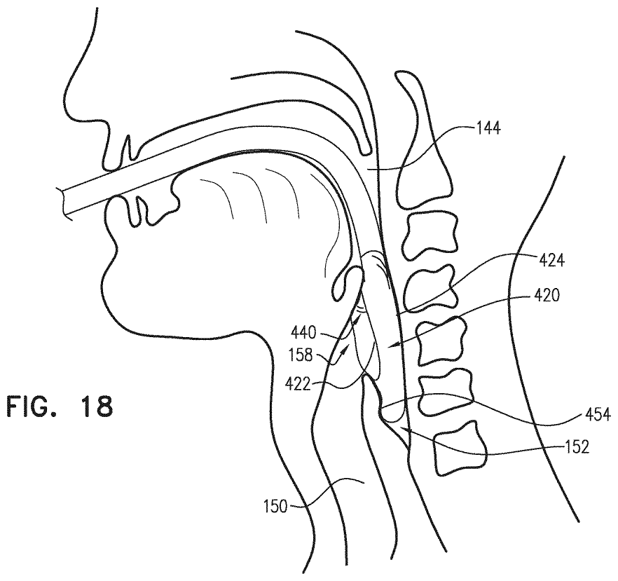

Reference is further made to FIG. 18, which is a schematic illustration of laryngeal mask airway device 420 disposed at insertion location 440, in accordance with an application of the present invention.

Inflatable annular cuff 432 is insertable through an anterior opening of an oral cavity of the patient. Cuff anterior side 422 of inflatable annular cuff 432 is configured to stabilize inflatable annular cuff 432 with respect to laryngeal inlet 158 of the patient when inflatable annular cuff 432 is disposed at insertion location 440 within throat 144 of the patient, as shown in FIG. 17A. Cuff anterior side 422 is shaped so at to define and surround a ventilation port 444.

Inflatable annular cuff 432 is also shaped so as to define a distal tip 450 that has a tip length T, measured between a distal end 452 of distal tip 450 and a proximal straight border 453 of distal tip 450, of at least 25 mm (e.g., at least 30 mm) and optionally less than 40 mm, when inflatable annular cuff 432 is filled with air at a pressure of 5 cm H2O and unconstrained, such that distal tip 450 forms a seal within an upper part 454 of esophagus 152 (optionally an upper esophageal sphincter) when inflatable annular cuff 432 is disposed at insertion location 440. Proximal straight border 453 of distal tip 450 is located at a longitudinal location 458 along inflatable annular cuff 432 at which inflatable annular cuff 432 has a width of 29 mm, measured from side to side of inflatable annular cuff 432.

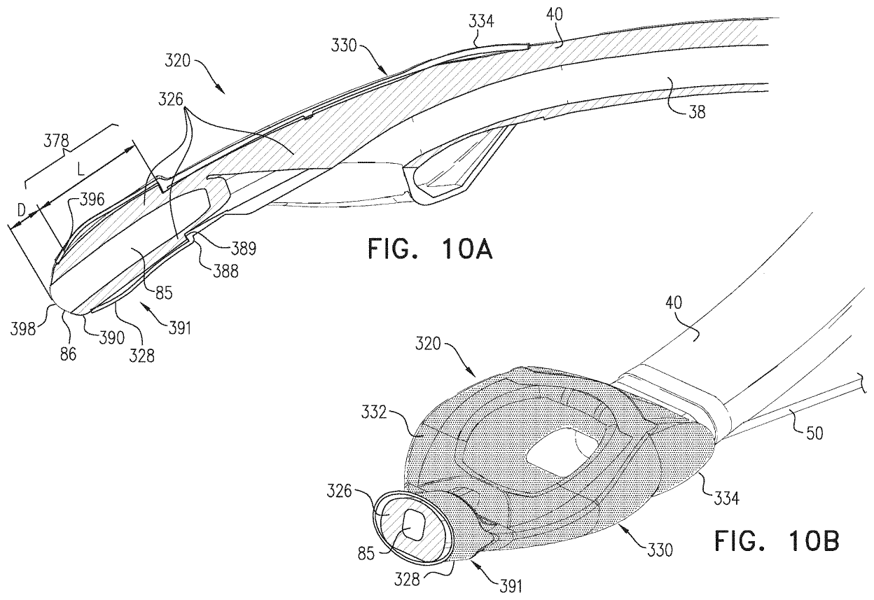

Inflatable annular cuffs of conventional laryngeal mask airway devices known in the art, such as shown in FIGS. 14A-B and 16A-B, typically have a tip length T, labeled in FIGS. 14A-B, of less than 20 mm, and thus form a seal as a lid on the opening of esophagus 152. By contrast, distal tip 450 of inflatable annular cuff 432 of the present invention penetrates inside upper part 454 of esophagus 152, typically to the level of the first or second tracheal cartilage rings immediately below the cricoid cartilage ring.

Reference is again made to FIGS. 15A-B, 17A-B, and 18. Laryngeal mask airway device 420 further comprises:
a shaft 460 having (a) a proximal end 462 that is configured to be disposed outside the patient's oral cavity when inflatable annular cuff 432 is disposed at insertion location 440, and (b) a distal end portion 464 that is fixed to inflatable annular cuff 432;
an airway tube 466, which passes through at least a longitudinal portion of shaft 460, and has (a) a proximal end 468 that is configured to be disposed outside the patient's oral cavity when inflatable annular cuff 432 is disposed at insertion location 440, and (b) a distal end 470 that is in fluid communication with ventilation port 444; and
an inflation tube 50 having (a) a proximal end 52 that is configured to be disposed outside the patient's oral cavity when inflatable annular cuff 432 is disposed at insertion location 440, and (b) a distal end 476 that is coupled in fluid communication with an interior of inflatable annular cuff 432.

Typically, cuff anterior side 422 is configured to form a seal around laryngeal inlet 158 when inflatable annular cuff 432 is disposed at insertion location 440.

For some applications, laryngeal mask airway device 420 further comprises a drainage tube 480 that (a) extends from a gastric channel 482 having a distal gastric opening 484 through distal tip 450, and (b) has a proximal drainage port at location outside the patient's oral cavity when inflatable annular cuff 432 is disposed at insertion location 440.

For some applications, inflatable annular cuff 432 comprises a highly elastic material, such as silicone, latex, or TPE. For other applications, inflatable annular cuff 432 comprises a material having minimal elasticity as commonly used in medical device cuff technology, e.g., PVC or polyurethane.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

In an embodiment, techniques and apparatus described in one or more of the following applications, which are assigned to the assignee of the present application and incorporated herein by reference, are combined with techniques and apparatus described herein:
U.S. Provisional App. No. 62/592,020, filed Nov. 29, 2017
U.S. Pat. No. 10,369,311 to Zachar
U.S. Pat. No. 10,173,022 to Zachar
U.S. Provisional App. No. 62/789,208, filed Jan. 7, 2019
PCT Publication WO 2019/106670 to Zachar
PCT Publication WO 2019/198081 to Zachar
U.S. Pat. No. 10,744,287 to Zachar
U.S. Provisional App. No. 63/035,057, filed Jun. 5, 2020
U.S. Provisional App. No. 63/079,639, filed Sep. 17, 2020
U.S. Provisional App. No. 63/171,516, filed Apr. 6, 2021

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A laryngeal mask airway device comprising:
an inflatable balloon, which is (a) shaped so as to define at least an inflatable annular cuff and a proximal inflatable pharyngeal portion, and (b) insertable through an anterior opening of an oral cavity of a patient, the inflatable annular cuff shaped so as to define a cuff anterior side that is configured to stabilize the inflatable annular cuff with respect to a laryngeal inlet of the patient when the inflatable balloon is disposed at an insertion location within a throat of the patient;
a shaft having (a) a proximal end that is configured to be disposed outside the patient's oral cavity when the inflatable balloon is disposed at the insertion location, and (b) a distal end portion that is fixed to the inflatable balloon;
an airway tube, which passes through at least a longitudinal portion of the shaft, and has (a) a proximal end that is configured to be disposed outside the patient's oral cavity when the inflatable balloon is disposed at the insertion location, and (b) a distal end that is open through the inflatable annular cuff, wherein the proximal inflatable pharyngeal portion of the inflatable balloon forms an air-tight seal with an external surface of the shaft; and an inflation tube having (a) a proximal end that is configured to be disposed outside the patient's oral cavity when the inflatable balloon is disposed at the insertion location, and (b) a distal end that is coupled in fluid communication with an inflatable chamber defined by the inflatable balloon, for supplying air to the inflatable chamber,
wherein the inflatable balloon is configured such that when the inflatable chamber is filled with air at a pressure of 10 cm H2O and unconstrained, a volume of the air in a proximal one-third of the inflatable chamber equals at least 200% of a volume of the air in a distal one-third of the inflatable chamber,
the proximal one-third of the inflatable chamber extending distally from a proximal end of the inflatable chamber along one third of a length of the inflatable chamber,
the distal one-third of the inflatable chamber extending proximally from a distal end of the inflatable chamber along one third of the length of the inflatable chamber, and
the length measured along a straight line segment connecting (a) a distal centroid of the distal end of the inflatable chamber and (b) a proximal centroid of the proximal end of the inflatable chamber.

2. The laryngeal mask airway device according to claim 1, wherein the inflatable balloon is configured such that when the inflatable chamber is filled with the air at the pressure of 10 cm H2O and unconstrained, the volume of the air in the proximal one-third of the inflatable chamber equals at least 300% of the volume of the air in the distal one-third of the inflatable chamber.

3. The laryngeal mask airway device according to claim 2, wherein the inflatable balloon is configured such that when the inflatable chamber is filled with the air at the pressure of 10 cm H2O and unconstrained, the volume of the air in the proximal one-third of the inflatable chamber equals at least 350% of the volume of the air in the distal one-third of the inflatable chamber.

4. The laryngeal mask airway device according to claim 3, wherein the inflatable balloon is configured such that when the inflatable chamber is filled with the air at the pressure of 10 cm H2O and unconstrained, the volume of the air in the proximal one-third of the inflatable chamber equals at least 400% of the volume of the air in the distal two-third of the inflatable chamber.

5. The laryngeal mask airway device according to claim 1, wherein the inflatable balloon is configured such that when the inflatable chamber is filled with air at a pressure of 20 cm H2O and unconstrained, the volume of the air in the proximal one-third of the inflatable chamber equals at least 200% of the volume of the air in the distal one-third of the inflatable chamber.

6. The laryngeal mask airway device according to claim 1, wherein the inflatable balloon is configured such that when the inflatable chamber is filled with air at a pressure of 25 cm H2O and unconstrained, the volume of the air in the proximal one-third of the inflatable chamber equals at least 200% of the volume of the air in the distal one-third of the inflatable chamber.

7. The laryngeal mask airway device according to claim 1, wherein the inflatable balloon is configured such that when the inflatable chamber is filled with the air at the pressure of 10 cm H2O and unconstrained, the volume of the air in the proximal one-third of the inflatable chamber is at least 5 cc.

8. The laryngeal mask airway device according to claim 7, wherein the inflatable balloon is configured such that when the inflatable chamber is filled with the air at the pressure of 10 cm H2O and unconstrained, the volume of the air in the proximal one-third of the inflatable chamber is at least 10 cc.

9. The laryngeal mask airway device according to claim 1, wherein the inflatable balloon and the shaft are configured such that the proximal inflatable pharyngeal portion of the inflatable balloon forms the air-tight seal entirely around a longitudinal portion of shaft.

10. The laryngeal mask airway device according to claim 1, the proximal inflatable pharyngeal portion extends in both lateral directions away from the straight line segment.

11. The laryngeal mask airway device according to claim 10, wherein the proximal inflatable pharyngeal portion is reflectionally symmetric about a plane of symmetry that extends in an anterior/posterior direction, wherein the straight line segment falls in the plane of symmetry.

12. The laryngeal mask airway device according to claim 1, wherein the cuff anterior side is configured to form a seal around the laryngeal inlet of the patient when the inflatable balloon is disposed at the insertion location.

13. The laryngeal mask airway device according to claim 1, wherein the inflation tube comprises an inflation valve that is configured to be disposed outside the patient's oral cavity when the inflatable balloon is disposed at the insertion location, and
wherein the laryngeal mask airway device is configured such that when the inflatable balloon, while the inflation valve is closed and the pressure within the inflatable balloon equals ambient pressure, is inserted from outside the oral cavity of the patient to the insertion location within the throat without opening the inflation valve, the pressure within the inflatable balloon increases from the ambient pressure to an inserted-location pressure, the inserted-location pressure less than 60 cm H2O.

14. The laryngeal mask airway device according to claim 13,
wherein the inserted-location pressure is less than 45 cm H2O.

15. The laryngeal mask airway device according to claim 14,
wherein the inserted-location pressure is less than 30 cm H2O.

16. The laryngeal mask airway device according to claim 1, wherein the inflatable annular cuff is shaped so as to define a distal tip portion that includes a distal end of the inflatable annular cuff,
wherein the distal tip portion is configured to form a seal against an upper
esophageal sphincter when the inflatable balloon is disposed at the insertion location, and wherein a distal wall of the distal tip portion has a thickness of at least 2 mm.

17. The laryngeal mask airway device according to claim 16, further comprising a drainage tube that (a) extends from a gastric channel having a distal gastric opening through the distal tip portion, and (b) has a proximal drainage port at location outside the patient's oral cavity when the inflatable balloon is disposed at the insertion location.

18. The laryngeal mask airway device according to claim 1, wherein the laryngeal mask airway device comprises a distal tip portion located distal to the inflatable annular cuff, and
wherein the distal tip portion is configured to form a seal against an upper esophageal sphincter when the inflatable balloon is disposed at the insertion location.

19-34. (canceled)

35. A laryngeal mask airway system comprising: laryngeal mask airway device, which comprises:
an inflatable balloon, which is shaped so as to define at least an inflatable annular cuff, which is insertable through an anterior opening of an oral cavity of a patient, the inflatable annular cuff shaped so as to define a cuff anterior side that is configured to stabilize the inflatable annular cuff with respect to a laryngeal inlet of the patient when the inflatable balloon is disposed at an insertion location within a throat of the patient;
a shaft having (a) a proximal end that is configured to be disposed outside the patient's oral cavity when the inflatable balloon is disposed at the insertion location, and (b) a distal end portion that is fixed to the inflatable balloon;
an airway tube, which passes through at least a longitudinal portion of the shaft, and has
(a) a proximal end that is configured to be disposed outside the patient's oral cavity when the inflatable balloon is disposed at the insertion location, and (b) a distal end that is open through the inflatable annular cuff; and
an inflation tube having (a) a proximal end that is configured to be disposed outside the patient's oral cavity when the inflatable balloon is disposed at the insertion location, and (b) a distal end that is coupled in fluid communication with an inflatable chamber defined by the inflatable balloon, for supplying air to the inflatable chamber; and
an insertion posterior protector, which comprises an elongate sheet of material that is shaped so as to define:
a proximal coupling portion, which is shaped so as to define one or more couplers that are configured to removably couple the proximal coupling portion to the shaft proximally to the inflatable balloon, and
a distal protection portion, which is shaped so as to partially cover a posterior external surface of the inflatable balloon when the proximal coupling portion is removably coupled to the shaft, so as to protect the inflatable balloon from puncture during insertion thereof into the patient's oral cavity.

36-58. (canceled)

59. A method comprising:
inserting an inflatable balloon of a laryngeal mask airway device through an anterior opening of an oral cavity of a patient, the inflatable balloon shaped so as to define at least an inflatable annular cuff and a proximal inflatable pharyngeal portion, and disposing the inflatable balloon at an insertion location within a throat of the patient, such that (a) a proximal end of an inflation tube is disposed outside the patient's oral cavity, wherein a distal end of the inflation tube is coupled in fluid communication with an inflatable chamber defined by the inflatable balloon, for supplying air to the inflatable balloon, (b) a proximal end of a shaft of the laryngeal mask airway device is disposed outside the patient's oral cavity, wherein a distal end portion of the shaft is fixed to the inflatable balloon, (c) a proximal end of an airway tube of the laryngeal mask airway device is disposed outside the patient's oral cavity, the airway tube passing through at least a longitudinal portion of the shaft, and having a distal end that is open through the inflatable annular cuff, wherein the proximal inflatable pharyngeal portion of the inflatable balloon forms an air-tight seal with an external surface of the shaft, and (d) a cuff anterior side of the inflatable annular cuff stabilizes the inflatable annular cuff with respect to a laryngeal inlet of the patient; and
ventilating lungs of the patient using the laryngeal mask airway device, wherein the inflatable balloon is configured such that when the inflatable chamber is filled with air at a pressure of 10 cm H2O and unconstrained, a volume of the air in a proximal one-third of the inflatable chamber equals at least 200% of a volume of the air in a distal one-third of the inflatable chamber,
the proximal one-third of the inflatable chamber extending distally from a proximal end of the inflatable chamber along one third of a length of the inflatable chamber,
the distal one-third of the inflatable chamber extending proximally from a distal end of the inflatable chamber along one third of the length of the inflatable chamber, and
the length measured along a straight line segment connecting (a) a distal centroid of the distal end of the inflatable chamber and (b) a proximal centroid of the proximal end of the inflatable chamber.

60-99. (canceled)
